(19) United States
(12) Patent Application Publication
(10) Pub. No.: US 2019/0292577 A1
(43) Pub. Date: Sep. 26, 2019

(54) COMPOSITIONS AND METHOD FOR THE TREATMENT OF ACNE

(71) Applicant: ProdermIQ, Inc., San Diego, CA (US)

(72) Inventors: Sasan AMINI, Redwood City, CA (US); Dana HOSSEINI, San Diego, CA (US)

(21) Appl. No.: 16/365,530

(22) Filed: Mar. 26, 2019

Related U.S. Application Data
(60) Provisional application No. 62/648,224, filed on Mar. 26, 2018.

(51) Int. Cl. C12Q 1/02; A61K 31/327; A61K 31/60; A61K 9/00; A61P 17/10; A61B 5/00; G06K 9/62; C12Q 1/04; C12Q 1/689

(52) U.S. Cl. CPC C12Q 1/025; A61K 31/327; A61K 31/60; A61K 9/0014; C12Q 2600/106; A61B 5/4848; G06K 9/6263; C12Q 1/04; C12Q 1/689; A61P 17/10

(57) ABSTRACT

Described herein are systems, methods, and compositions for treating a skin condition in an individual. A system or method is used to determine a skin flora of an individual and a sensitivity of the skin flora to a treatment modality. A composition is then customized based on the sensitivity of the skin flora to the treatment modality.

### CROSS REFERENCE

This application claims the benefit of priority from U.S. Provisional Patent Application No. 62/648,224, filed Mar. 26, 2018, which is incorporated herein by reference in its entirety.

### BACKGROUND

The pathology of acne is known to relate to sebaceous gland activity, Propionibacterium acnes (P. acnes) colonization, obstruction of the sebaceous follicle, and inflammation. The importance of P. acnes colonization as a prerequisite for early preadolescent acne has been debated.

### SUMMARY

Described herein are systems and methods for determining the microbiome present on a skin surface of an individual from a cohort of individuals and, in some embodiments, further determining the sensitivity of a flora of the microbiome to a treatment modality for treating an abnormality associated with the skin of the individual. A treatment modality as used herein includes topical formulations for treatment. An abnormality on the surface of the skin of an individual as used herein includes acne on the skin surface of the individual.

Described herein are systems and methods for determining a custom composition for treating a skin surface of an individual affected by an abnormality. In some embodiments, systems and methods described herein are configured to treat acne in an individual based on the microflora on the skin surface of the individual and/or a sensitivity of the microflora (or a subset of the flora of the microflora) to a treatment including but not limited to benzoyl peroxide.

Described herein are compositions for treating a skin condition in an individual including compositions for treating acne vulgaris.

There is significant diversity among the microbiomes of different individuals in terms of the flora and also strains of flora within a microbiome of an individual. In the case of certain conditions such as acne (i.e., acne vulgaris), certain specific flora are believed to be associated with or possibly causative of the skin condition. P. acnes in some individuals is associated with the condition of acne and treatment of P. acnes in these individuals treats or prevents acne formation.

Described herein are systems and methods for determining the presence of P. acnes in the skin flora of an individual and determining the sensitivity of P. acnes to a treatment.

In some embodiments, a treatment for which the sensitivity of P. acnes is determined is the administration of benzoyl peroxide.

Described herein are findings of a study of individuals comprising preadolescent females with acne, and changes in the microbiomes of the individuals with the use of benzoyl peroxide wash. Flora was taken from different areas of the faces of the individuals and studied for sensitivity to benzoyl peroxide.

Described herein is a method for treating a skin condition in an individual, said method comprising: obtaining a sample from said individual; identifying a presence of a micro-organism within said sample; identifying a strain of said micro-organism associated with a therapeutic response to a treatment; and treating said skin condition with said treatment. In some embodiments, said skin condition comprises acne vulgaris. In some embodiments, said micro-organism comprises P. acnes. In some embodiments, said presence of said micro-organism is identified using genome sequencing on said sample. In some embodiments, said strain of said micro-organism is associated with a therapeutic response to said treatment based on an analysis of a metabolism of said micro-organism. In some embodiments, said treatment comprises benzoyl peroxide.

Described herein is a method for treating acne vulgaris in an individual, said method comprising: obtaining a sample from said individual; and identifying a strain of P. acnes associated with a therapeutic response to benzoyl peroxide. In some embodiments, the method comprises providing benzoyl peroxide to said individual. In some embodiments, said strain of P. acnes associated with a therapeutic response to benzoyl peroxide is identified using genomic sequencing. In some embodiments, said strain of P. acnes associated with a therapeutic response to benzoyl peroxide is identified based on an analysis of a metabolism of said strain.

Described herein is a method of treating acne vulgaris in an individual, said method comprising: providing benzoyl peroxide to said individual; wherein a microbiome of a skin surface of said individual is found to comprise a strain of P. acnes associated with a therapeutic response to benzoyl peroxide. In some embodiments, said strain comprises a K4 strain. In some embodiments, said strain comprises an A2 strain. In some embodiments, said therapeutic response is determined using a machine learning algorithm.

Described herein is a method for analyzing a therapeutic efficacy for a skin condition in an individual, said method comprising: obtaining a sample from said individual; determining a presence of said micro-organism within said sample; determining a first degree of sensitivity of said micro-organism to a first treatment; identifying a second degree of sensitivity of said micro-organism to said second treatment; comparing said first degree of sensitivity to said second degree of sensitivity thereby generating a comparison result; and ranking said first treatment relative to said second treatment based on said comparison result. In some embodiments, said skin condition comprises acne vulgaris. In some embodiments, said micro-organism comprises P. acnes. In some embodiments, said presence of said micro-organism is determined using genome sequencing on said sample. In some embodiments, said strain of said micro-organism is associated with a therapeutic response to said first treatment and said second treatment based on an analysis of a metabolism of said micro-organism. In some embodiments, said first treatment comprises benzoyl peroxide.

Described herein is a computer based method for treating acne vulgaris in an individual, said method comprising: obtaining a sample from said individual; identifying a strain of P. acnes within said sample; determining using a machine learning model that said strain of P. acnes is associated with a therapeutic response to benzoyl peroxide; and treating said individual with said benzoyl peroxide when said strain of P. acnes is associated with said therapeutic response to said benzoyl peroxide. In some embodiments, the machine learning model is generated by a convolutional neural network. In some embodiments, the convolutional neural network is trained using supervised learning of labeled sample data taken from other individuals. In some embodiments, the therapeutic response to benzoyl peroxide is determined using genomic sequencing of said strain of P. acnes. In some embodiments, the strain of P. acnes associated with a therapeutic response to benzoyl peroxide is identified based on an analysis of a metabolism of said strain of P. acnes. In some embodiments, the therapeutic response to benzoyl peroxide comprises a decrease of at least 10% in a population of said P. acnes within a treatment area. In some embodiments, the strain of P. acnes is associated with a therapeutic response to benzoyl peroxide with an accuracy of at least 75%. In some embodiments, the strain of P. acnes comprises a K4 strain. In some embodiments, the strain said strain of P. acnes comprises an A2 strain.

Described herein is a method of treating acne vulgaris in an individual, said method comprising: providing benzoyl peroxide to said individual when a microbiome of a skin surface of said individual is found to comprise a strain of P. acnes selected from the group consisting of a K4 strain and an A2 strain. In some embodiments, the method additionally comprises providing salicylic acid to said individual. In some embodiments, the strain of P. acnes is associated with a therapeutic response to benzoyl peroxide. In some embodiments, the method additionally comprises a metabolism of said strain of P. acnes and wherein said therapeutic response to benzoyl peroxide is based on said metabolism. In some embodiments, the method additionally comprises instructing said individual to apply said benzoyl peroxide to an area of skin of said individual. In some embodiments, a population of P. acnes is reduced by at least 50% on said area of said skin after said benzoyl peroxide is applied.

Described herein is a method for analyzing a therapeutic efficacy of a treatment for acne vulgaris in an individual, said method comprising: obtaining a sample from said individual; determining a presence of P. acnes within said sample; determining a sensitivity of said P. acnes to each one of a plurality of treatments; ranking said each one of said plurality of treatments based on said sensitivity thereby identifying a most effective treatment and a second most effective treatment; and generating a combination therapy that includes said most effective treatment and said second most effective treatment. In some embodiments, the presence of said P. acnes is determined using genome sequencing on said sample. In some embodiments, the sensitivity of said strain of said P. acnes is determined based on an analysis of a metabolism of said micro-organism. In some embodiments, the sensitivity of said strain of said P. acnes is determined by a machine learning model that is generated by a convolutional neural network. In some embodiments, the convolutional neural network is trained using supervised learning of labeled sample data taken from other individuals.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a graph of the predicted flora on the skin surface of an individual affected by acne where the predicted flora are represented on the x-axis and the Mean Decrease Gini is represented on the y-axis.

FIG. 2 shows flora diversity at different locations on the skin surfaces of two different individuals Shannon and Simpson.

FIG. 3 shows multiple graphs of subsets of microbiomes in which benzoyl peroxide was applied and those where no benzoyl peroxide was applied and shows the relative proportion of the flora in each group of benzoyl peroxide and no benzoyl peroxide

FIG. 4 shows Bray-Curtis site specific results.

FIG. 5 shows a graph of species used to determine whether a skin region was exposed to benzoyl peroxide or no benzoyl peroxide.

FIG. 6 shows a graph of species used to determine whether a skin region comprising a comedone was exposed to benzoyl peroxide or no benzoyl peroxide.

FIG. 7 shows a graph of species used to determine whether a skin region comprising a forehead was exposed to benzoyl peroxide or no benzoyl peroxide.

FIG. 8 shows a graph of species used to determine whether a skin region comprising the outside of the nose was exposed to benzoyl peroxide or no benzoyl peroxide.

FIG. 9 shows a graph of species used to determine whether a skin region comprising behind the ear was exposed to benzoyl peroxide or no benzoyl peroxide.

FIG. 10 shows a comparative functional analysis for flora on skin regions to which benzoyl peroxide was applied and flora on skin regions to which no benzoyl peroxide was applied.

FIG. 11 shows a comparative functional analysis for flora on the cheek to which benzoyl peroxide was applied and flora on the cheek to which no benzoyl peroxide was applied.

FIG. 12 is a representation of the Bray Curtis Dissimilarity for the cheek skin region.

FIG. 13 shows a representation of total diversity at genus level for site specific and treatment specific results (i.e., with benzoyl peroxide and with no benzoyl peroxide).

FIG. 14 shows a representation of total diversity at the species level for site specific and treatment specific results (i.e. with benzoyl peroxide and with no benzoyl peroxide).

FIG. 15 shows a comparison of statistically significant taxa between two different samples taken from different times (visits) where samples were taken from the cheek.

FIG. 16 shows diversity measures both before and after benzoyl peroxide is added to a skin region.

FIG. 17 shows taxonomic genera with a significant change between two different visits at a species level.

FIG. 18 shows taxonomic genera with a significant change between two different visits at a genus level.

FIG. 19 shows diversity measurement results at two different visits of an individual.

FIG. 20 shows comparative microbiome profiling based on clinical outcome.

FIG. 21 shows the top 20 taxa that can differentiate the two visits for which data is shown herein in the cohort termed the “Decrease Cohort.”

FIG. 22 shows the top 20 taxa that can differentiate the two visits for which data is shown herein in the cohort termed the “Increase Cohort.”

FIG. 23 shows NMDS (Non-metric Multi-Dimensional Scaling) results for the “Decrease Cohort” across two visits.

FIG. 24 shows NMDS results for a “Flat Cohort” across two visits.

FIG. 25 shows NMDS results for the “Increase Cohort” across two visits.

FIG. 26 shows NMDS results for all outcomes across two visits.

FIG. 27 shows results of a comparison between the increase and decrease cohort.

FIG. 28 shows alpha diversity across sites based on treatment impact.

FIG. 29 shows alpha diversity across sites based on visits.

FIG. 30 shows the result of comparing statistically significant taxa in Increase and Decrease Cohorts for the cheek site.

FIG. 31 shows the result of comparing statistically significant taxa in the Increase and Decrease Cohorts for the forehead site.

FIG. 32 shows a graph representing the flora that are the top predictors for providing a prediction of a response to benzoyl peroxide based on the microbiome of the individual.

FIG. 33 shows a representation of P. acnes across different groups.

FIG. 34 shows P. acnes strains differentiated between Increase and Decrease Cohorts in the area outside of the nose.

FIG. 35 shows P. acnes strains differentiated between Increase and Decrease Cohorts in the area of the cheek.

FIG. 36 shows P. acnes strains differentiated between Increase and Decrease Cohorts in the area of the forehead.

FIG. 37 shows P. acnes strains differentiated before and after benzoyl peroxide treatment of the forehead.

FIG. 38 shows P. acnes strains differentiated between Increase & Decrease Cohorts.

FIG. 39 shows a change in P. acnes strains after treatment. The C2 P. acnes strain becomes more prevalent after benzoyl peroxide treatment.

FIG. 40 shows a comparative functional analysis based on response. A comparative functional analysis was done between those who responded to benzoyl peroxide and those who did not.

FIG. 41 shows a change in P. acnes strains after benzoyl peroxide treatment. Functional mapping of P. acnes strains shows significant differences in lipase activity, antibiotic resistance, siderophores, and peptides/nickel transport system.

FIG. 42 shows functional results of Manganese oxidation. Manganese oxidation is over-represented in the cohort that did not respond to benzoyl peroxide.

FIG. 43 shows an exemplary method for acne product recommendation.

FIG. 44 shows result of metabolic modeling of good and bad strains based on different metabolites.

FIG. 45 shows the results of acne product recommendation based on metabolic modeling.

FIG. 46 shows a benzoyl peroxide uptake model based on microbiome profiles.

FIG. 47 shows the results of mapping metabolic models to strains differentially represented in benzoyl peroxide response groups.

FIG. 48 shows a summary of the results of product recommendation proof of concept performed.

FIG. 49 shows the steps of an exemplary method from testing of an individual to the development of a custom treatment.

FIG. 50 shows the results of comprehensive biomarkers compiled for acne based on clinical data.

FIG. 51 shows a ranking of acne treatments.

FIG. 52 shows a schematic representation of an algorithm for modeling a Genome-Scale Metabolic reconstruction.

FIG. 53 shows a schematic representation of an exemplary computer based system.

### DETAILED DESCRIPTION

Commensal and mutualistic organisms help keep our bodies healthy in many ways: they help us to digest foods and acquire nutrients such as vitamins B and K, encourage the immune system to develop and prevent the colonization of, for example, bacterial pathogens that cause disease by competing with them. Together all of the microorganisms living in and on the body of an individual—commensal, mutualistic and pathogenic—are referred to as the microbiome. The metabolic processes and/or the products of the metabolic processes of the organisms that comprise the microbiome of the body of an individual are referred to as a metabolome. The equilibrium of organisms within the microbiome and the metabolome associated with these organisms that comprise the microbiome are closely linked to an individual's health status and vice-versa.

Described herein are systems and methods for generating customized skin care and personal care products for human and animal use and, more particularly, but not by way of limitation, to the development of personal care products that are based on the initial evaluation of the flora and/or metabolic activity of the flora inhabiting the skin and subcutaneous tissue.

Described herein are systems and methods for analyzing the skin and subcutaneous tissue flora, e.g., the microbiome, and its associated metabolome, comparing the resulting profile of the skin and subcutaneous tissue flora and metabolome to a healthy profile, represented as a quantity and diversity of flora that falls within a range determined from a set of healthy skin types and/or unhealthy skin types, and then customizing skin care and personal care products that will augment the flora residing on a test subject's skin and subcutaneous tissue and its associated metabolome or replicate a healthy flora profile on to that of a test subject.

Next generation sequencing (NGS) has created an opportunity to quickly and accurately identify and profile the microbiome inhabiting the skin and subcutaneous tissue, which then creates an opportunity for the creation of customized or personalized skin care and personal care products that either maintain a healthy microbiome or shift a profile towards a healthy equilibrium or profile by blending a mixture of commensal and/or mutualistic organisms specifically created to establish a healthy profile. The optimal flora also interacts with the host immune system in a synergistic way further propagating its health benefits. The associated metabolome of individuals can also be profiled either by a mass-spectrometry based system or using genomics-based metabolome modeling and flux-balance analysis and used to make a healthy metabolome profile. Deficiencies in any of the beneficial metabolites can be supplemented as well.

Traditional treatments of certain dermatological conditions comprise antibiotics that drastically impact the microbiome including the commensal and mutualistic bacteria. Other traditional treatments of certain dermatological conditions comprise anti-inflammatory agents such as steroids that have local and systemic effects on immune response. Both of these traditional treatments, antibiotic and steroid based therapies, may fail to address the underlying cause of a skin condition if it is due to an imbalance or absence of commensal or mutualistic microorganisms, overabundance of opportunistic or pathogenic bacteria, or deficiencies of essential or beneficial metabolites.

Described herein is a computer based method for treating acne vulgaris in an individual, said method comprising: obtaining a sample from said individual; identifying a strain of P. acnes within said sample; determining using a machine learning model that said strain of P. acnes is associated with a therapeutic response to benzoyl peroxide; and treating said individual with said benzoyl peroxide when said strain of P. acnes is associated with said therapeutic response to said benzoyl peroxide. In some embodiments, the machine learning model is generated by a convolutional neural network. In some embodiments, the convolutional neural network is trained using supervised learning of labeled sample data taken from other individuals. In some embodiments, the therapeutic response to benzoyl peroxide is determined using genomic sequencing of said strain of P. acnes. In some embodiments, the strain of P. acnes associated with a therapeutic response to benzoyl peroxide is identified based on an analysis of a metabolism of said strain of P. acnes. In some embodiments, the therapeutic response to benzoyl peroxide comprises a decrease of at least 10% in a population of said P. acnes within a treatment area. In some embodiments, the strain of P. acnes is associated with a therapeutic response to benzoyl peroxide with an accuracy of at least 75%. In some embodiments, the strain of P. acnes comprises a K4 strain. In some embodiments, the strain said strain of P. acnes comprises an A2 strain.

Described herein is a method of treating acne vulgaris in an individual, said method comprising: providing benzoyl peroxide to said individual when a microbiome of a skin surface of said individual is found to comprise a strain of P. acnes selected from the group consisting of a K4 strain and an A2 strain. In some embodiments, the method additionally comprises providing salicylic acid to said individual. In some embodiments, the strain of P. acnes is associated with a therapeutic response to benzoyl peroxide. In some embodiments, the method additionally comprises a metabolism of said strain of P. acnes and wherein said therapeutic response to benzoyl peroxide is based on said metabolism. In some embodiments, the method additionally comprises instructing said individual to apply said benzoyl peroxide to an area of skin of said individual. In some embodiments, a population of P. acnes is reduced by at least 50% on said area of said skin after said benzoyl peroxide is applied.

Described herein is a method for analyzing a therapeutic efficacy of a treatment for acne vulgaris in an individual, said method comprising: obtaining a sample from said individual; determining a presence of P. acnes within said sample; determining a sensitivity of said P. acnes to each one of a plurality of treatments; ranking said each one of said plurality of treatments based on said sensitivity thereby identifying a most effective treatment and a second most effective treatment; and generating a combination therapy that includes said most effective treatment and said second most effective treatment. In some embodiments, the presence of said P. acnes is determined using genome sequencing on said sample. In some embodiments, the sensitivity of said strain of said P. acnes is determined based on an analysis of a metabolism of said micro-organism. In some embodiments, the sensitivity of said strain of said P. acnes is determined by a machine learning model that is generated by a convolutional neural network. In some embodiments, the convolutional neural network is trained using supervised learning of labeled sample data taken from other individuals.

### Certain Terminology

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein, the terms “sample” and “biological sample” refer to any sample suitable for the methods provided by the present invention. A sample of cells can be any sample, including, for example, a skin or subcutaneous tissue sample obtained by non-invasive techniques such as tape stripping, scraping, swabbing, or more invasive techniques such as biopsy of a subject. In one embodiment, the term “sample” refers to any preparation derived from skin or subcutaneous tissue of a subject. For example, a sample of cells obtained using the non-invasive method described herein can be used to isolate nucleic acid molecules or proteins for the methods of the present invention. Samples for the present invention may be taken from an area of the skin shown to exhibit a disease or disorder, which is suspected of being the result of a disease or a pathological or physiological state, such as psoriasis or dermatitis, or the surrounding margin or tissue. As used herein, “surrounding margin” or “surrounding tissue” refers to tissue of the subject that is adjacent to the skin shown to exhibit a disease or disorder, but otherwise appears to be normal.

In some embodiments, the method further includes comparing the microbiome of the subject to a reference microbiome or generating a microbiome profile of the subject, or identifying a disease or disorder which the subject has, or is at risk of developing, or providing a personalized treatment regime to the subject. In various embodiments, the reference microbiome is classified as having a healthy profile and a similarity between the microbiome of the subject and the reference microbiome identifies the microbiome of the subject as having a healthy profile. Alternatively, the reference microbiome is classified as having, or at risk of having a disease or disorder and a similarity between the microbiome of the subject and the reference microbiome identifies the microbiome of the subject as having as having, or at risk of having the disease or disorder.

As used herein “a healthy profile” refers to a microbiome associated with a sample from a subject that is from a subject that is determined to have a healthy microbiome, e.g., free from disease or disorder, or risk thereof. As such, in one embodiment, the reference microbiome is that of a sample of cells obtained from a healthy individual that does not have a skin disorder or particular undesirable phenotype.

The term healthy profile refers a quantity and diversity of flora that falls within a range determined from a set of healthy skin types. The term healthy skin refers to skin that is devoid of a skin condition, disease or disorder, including, but not limited to inflammation, rash, dermatitis, atopic dermatitis, eczema, psoriasis, dandruff, acne, cellulitis, rosacea, warts, seborrheic keratosis, actinic keratosis, tinea versicolor, viral exantham, shingles, ringworm, and cancer, such as basal cell carcinoma, squamous cell carcinoma, and melanoma.

Additionally, as used herein, a “disease” or “disorder” is intended to generally refer to any skin associated disease, for example, but in no way limited to, inflammation, rash, dermatitis, atopic dermatitis, eczema, psoriasis, dandruff, acne, cellulitis, rosacea, warts, seborrheic keratosis, actinic keratosis, tinea versicolor, viral exantham, shingles, ringworm, and cancer, such as basal cell carcinoma, squamous cell carcinoma, and melanoma.

The term “cancer” as used herein, includes any malignant tumor including, but not limited to, carcinoma, melanoma and sarcoma. Cancer arises from the uncontrolled and/or abnormal division of cells that then invade and destroy the surrounding tissues. As used herein, “proliferating” and “proliferation” refer to cells undergoing mitosis. As used herein, “metastasis” refers to the distant spread of a malignant tumor from its sight of origin. Cancer cells may metastasize through the bloodstream, through the lymphatic system, across body cavities, or any combination thereof. The term “cancerous cell” as provided herein, includes a cell afflicted by any one of the cancerous conditions provided herein. The term “carcinoma” refers to a malignant new growth made up of epithelial cells tending to infiltrate surrounding tissues, and to give rise to metastases. The term “melanoma” refers to a malignant tumor of melanocytes which are found predominantly in skin but also in bowel and the eye. “Melanocytes” refer to cells located in the bottom layer, the basal lamina, of the skin's epidermis and in the middle layer of the eye. Thus, “melanoma metastasis” refers to the spread of melanoma cells to regional lymph nodes and/or distant organs (e.g., liver, brain, breast, prostate, etc.).

The term “skin” or “subcutaneous tissue” refers to the outer protective covering of the body, consisting of the epidermis (including the stratum corneum) and the underlying dermis, and is understood to include sweat and sebaceous glands, as well as hair follicle structures and nails. Throughout the present application, the adjective “cutaneous” and “subcutaneous” can be used, and should be understood to refer generally to attributes of the skin, as appropriate to the context in which they are used. The epidermis of the human skin comprises several distinct layers of skin tissue. The deepest layer is the stratum basalis layer, which consists of columnar cells. The overlying layer is the stratum spinosum, which is composed of polyhedral cells. Cells pushed up from the stratum spinosum are flattened and synthesize keratohyalin granules to form the stratum granulosum layer. As these cells move outward, they lose their nuclei, and the keratohyalin granules fuse and mingle with tonofibrils. This forms a clear layer called the stratum lucidum. The cells of the stratum lucidum are closely packed. As the cells move up from the stratum lucidum, they become compressed into many layers of opaque squamae. These cells are all flattened remnants of cells that have become completely filled with keratin and have lost all other internal structure, including nuclei. These squamae constitute the outer layer of the epidermis, the stratum corneum. At the bottom of the stratum corneum, the cells are closely compacted and adhere to each other strongly, but higher in the stratum they become loosely packed, and eventually flake away at the surface.

A “probe” or “probe nucleic acid molecule” is a nucleic acid molecule that is at least partially single-stranded, and that is at least partially complementary, or at least partially substantially complementary, to a sequence of interest. A probe can be RNA, DNA, or a combination of both RNA and DNA. It is also within the scope of the present invention to have probe nucleic acid molecules comprising nucleic acids in which the backbone sugar is other that ribose or deoxyribose. Probe nucleic acids can also be peptide nucleic acids. A probe can comprise nucleolytic-activity resistant linkages or detectable labels, and can be operably linked to other moieties, for example a peptide.

A single-stranded nucleic acid molecule is “complementary” to another single-stranded nucleic acid molecule when it can base-pair (hybridize) with all or a portion of the other nucleic acid molecule to form a double helix (double-stranded nucleic acid molecule), based on the ability of guanine (G) to base pair with cytosine (C) and adenine (A) to base pair with thymine (T) or uridine (U). For example, the nucleotide sequence 5′-TATAC-3′ is complementary to the nucleotide sequence 5′-GTATA-3′.

As used herein “hybridization” refers to the process by which a nucleic acid strand joins with a complementary strand through base pairing. Hybridization reactions can be sensitive and selective so that a particular sequence of interest can be identified even in samples in which it is present at low concentrations. In an in vitro situation, suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature. For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 mg/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

As used herein, the term “skin flora” or “microbiome” refers to microorganisms, including bacteria, viruses, and fungi that inhabit the skin or subcutaneous tissues of the subject.

As used herein, the terms microbial, microbe, or microorganism refer to any microscopic organism including prokaryotes or eukaryotes, bacterium, archaebacterium, fungus, virus, or protist, unicellular or multicellular.

As used herein, the term “ameliorating” or “treating” means that the clinical signs and/or the symptoms associated with the cancer or melanoma are lessened as a result of the actions performed. The signs or symptoms to be monitored will be characteristic of a particular cancer or melanoma and will be well known to the skilled clinician, as will the methods for monitoring the signs and conditions. Thus, a “treatment regimen” refers to any systematic plan or course for treating a disease or cancer in a subject.

In embodiments, nucleic acid molecules can also be isolated by lysing the cells and cellular material collected from the skin sample by any number of means well known to those skilled in the art. For example, a number of commercial products available for isolating polynucleotides, including but not limited to, RNeasy™ (Qiagen, Valencia, Calif.) and TriReagent™ (Molecular Research Center, Inc, Cincinnati, Ohio) can be used. The isolated polynucleotides can then be tested or assayed for particular nucleic acid sequences, including a polynucleotide encoding a cytokine. Methods of recovering a target nucleic acid molecule within a nucleic acid sample are well known in the art, and can include microarray analysis.

As used herein, the term “commensal microorganisms” means both prokaryotes and eukaryotes that may colonize (i.e., live and multiply on human skin) or temporarily inhabit human skin in vitro, ex vivo and/or in vivo. Exemplary skin commensal microorganisms include, but are not limited to, Alphaproteobacteria, B etaproteobacteria, Gammaproteobacteria, Propionibacteria, Corynebacteria, Actinobacteria, Clostridiales, Lactobacillales, Staphylococcus, Bacillus, Micrococcus, Streptococcus, Bacteroidales, Flavobacteriales, Enterococcus, Pseudomonas, Malassezia, Maydida, Debaroyomyces, and Cryptococcus.

### Systems and Methods for Microbiome Determination

Described herein are systems and methods that characterize a skin microbiome of an individual. The process of characterization described herein includes an analysis of the microbial flora and/or associated metabolome of a sample taken from, for example, the skin of an individual. In some embodiments, the process of characterization comprises determining if a condition of the tissue (e.g. a disease or disorder) is a result of an imbalance or absence of commensal or mutualistic microorganisms and/or an imbalance or deficiency in the associated metabolome.

Described herein are systems and methods for analyzing samples taken from individuals having certain disorders and diseases in order to characterize the sample, and, in some embodiments, provide a custom therapy to the individuals based on the characterization. More specifically, analysis is performed on the samples to characterize the microbiome and/or metabolome data associated with the sample in terms of: (a) the taxonomy of micro-organisms that comprise the microbiome, (b) the metabolome profile associated with the microbiome, and/or (c) the physical expression of the microbiome and/or metabolome in the individual. For example, in some embodiments of the systems and methods described herein, a percentage of different bacteria are identified within a sample and an imbalance with respect to the individual's microbiome is detected in the form of overgrowth of a species of micro-organism that is typically in low numbers in the microbiome of normal individuals (or in this individual in a non-diseased state). In another example, a metabolome profile is determined in terms of identifying the percentage of metabolites present in a sample taken from an individual and detecting an imbalance in terms of an overproduction of a certain metabolite that is typically in low numbers in normal individuals (or in this individual in a non-diseased state). In an additional example, a physical expression of the microbiome and/or metabolome is identified in the individual by comparing the microbiome and/or metabolome characteristics of the individual to those of normal individuals (or the same individual in a non-diseased state). That is, in some embodiments, a physical expression of the microbiome and/or metabolome of the individual indicates that they have a high amount of body odor based on a comparison of the characteristics of the microbiome and/or metabolome of the individual with the microbiomes and/or metabolomes of others. In this way, an individual is classified. In this specific example, an individual is classified as having a high amount of body odor.

Characterization of a sample taken from an individual, in some embodiments, is based on a comparison of the sample analysis results of one individual to those of one or more health individuals. Healthy individuals provide samples or sample analysis data that is determined to have a healthy microbiome, e.g., free from disease or disorder, or risk thereof and/or is free of a particular disease or disorder. As such, in some embodiments, a reference microbiome is taken from one or more samples of cells obtained from one or more healthy individuals that do not have a skin disorder and/or particular undesirable phenotype. Likewise, a healthy profile comprises a quantity and diversity of flora that falls within a range determined from a set of healthy skin types. The term healthy skin comprises skin that is devoid of a skin condition, disease or disorder, including, but not limited to inflammation, rash, dermatitis, atopic dermatitis, eczema, psoriasis, dandruff, acne, cellulitis, rosacea, warts, seborrheic keratosis, actinic keratosis, tinea versicolor, viral exantham, shingles, ringworm, and cancer, such as basal cell carcinoma, squamous cell carcinoma, and melanoma. The systems and methods described herein, in classifying individuals based on sample analysis, also provide the diagnosis of diseases and disorders in certain individuals. Non-limiting examples of diseases and disorders diagnosed by embodiments of the systems and methods described herein include inflammation, rash, dermatitis, atopic dermatitis, eczema, psoriasis, dandruff, acne, cellulitis, rosacea, warts, seborrheic keratosis, actinic keratosis, tinea versicolor, viral exantham, shingles, ringworm, and cancer, such as basal cell carcinoma, squamous cell carcinoma, melanoma, carcinoma, and sarcoma.

Samples suitable for use with the systems and methods described herein include a skin or subcutaneous tissue sample obtained by non-invasive techniques such as tape stripping, scraping, swabbing, or more invasive techniques such as biopsy of a subject. It should be understood that samples suitable for use with the systems and methods described herein include any preparation derived from the skin or subcutaneous tissue of an individual. Likewise, samples suitable for use with the systems and methods described herein, in some embodiments, are taken from an area of the skin shown to exhibit a disease or disorder, which is suspected of being the result of a disease or a pathological or physiological state, such as psoriasis or dermatitis, or the surrounding margin or tissue. Likewise, samples taken from a surrounding margin or surrounding tissue refers to tissue of the subject that is adjacent to the skin shown to exhibit a disease or disorder, but otherwise appears to be normal and these types of samples are also suitable for use with the systems and methods described herein. The skin and subcutaneous tissue comprise the outer protective covering of the body, and comprise the epidermis (including the stratum corneum) and the underlying dermis, and is understood to include sweat and sebaceous glands as well as hair follicle structures and nails. Throughout the present application, the adjective “cutaneous” and “subcutaneous” can be used, and should be understood to refer generally to attributes of the skin, as appropriate to the context in which they are used. The epidermis of the human skin comprises several distinct layers of skin tissue. The deepest layer is the stratum basalis layer, which consists of columnar cells. The overlying layer is the stratum spinosum, which is composed of polyhedral cells. Cells pushed up from the stratum spinosum are flattened and synthesize keratohyalin granules to form the stratum granulosum layer. As these cells move outward, they lose their nuclei, and the keratohyalin granules fuse and mingle with tonofibrils. This forms a clear layer called the stratum lucidum. The cells of the stratum lucidum are closely packed. As the cells move up from the stratum lucidum, they become compressed into many layers of opaque squamae. These cells are all flattened remnants of cells that have become completely filled with keratin and have lost all other internal structure, including nuclei. These squamae constitute the outer layer of the epidermis, the stratum corneum. At the bottom of the stratum corneum, the cells are closely compacted and adhere to each other strongly, but higher in the stratum they become loosely packed, and eventually flake away at the surface.

As discussed further herein, Next Generation Sequencing, or “NGS”, is a powerful DNA sequencing technology that allows for the rapid and accurate sequencing of cells or organisms, and enables evaluating complex bacterial communities, a good example of which is the microbiome. In some embodiments, identification of inhabitant flora for every individual is conducted on such an NGS platform. Such a platform allows for the rapid and accurate generation of a profile of the microbiome inhabiting the skin of an individual with high enough sensitivity and specificity with a relatively short turn-around time and scalable throughput.

Alternatively, a Sanger-sequencing, mass-spectrometry, quantitative PCR, immunofluorescence, in situ hybridization, or microbial staining based platform can be used to characterize individual profiles. Similarly, the microbiome or metabolome can be profiled either by a mass-spectrometry based system or using genomics-based metabolome modeling and flux-balance analysis. All the above-mentioned identification methods can be implemented on samples directly collected from individuals without any proliferation step. This way, minimal bias is introduced toward identification of a mixture of culturable and unculturable microorganisms or their associated metabolome.

By leveraging the high throughput capabilities of NGS or other microbial identification methods like mass spectrometry or Sanger sequencing, microorganisms on an individual's subcutaneous tissue and their associated microbiome and metabolome will simultaneously be identified and the resulting profile may be compared to a healthy profile from a database of skin and subcutaneous tissue profiles. Independent of which platform is exploited for profiling, the abovementioned platform may be offered as a test to any client and the output may be used to identify which commensal, pathogenic, or mutualistic microorganisms or their associated metabolite are depleted or overrepresented on the subject's skin and subcutaneous tissue compared to the healthy profile.

Probes suitable for use with the systems and methods described herein comprise nucleic acid molecule that are at least partially single-stranded, and that are at least partially complementary, or at least partially substantially complementary, to a sequence of interest. A probe can be RNA, DNA, or a combination of both RNA and DNA. Suitable probes also comprise nucleic acid molecules comprising nucleic acids in which the backbone sugars other than ribose or deoxyribose. Suitable probes also comprise nucleic acids comprising peptide nucleic acids. A probe in some embodiments comprises nucleolytic-activity resistant linkages or detectable labels, and can be operably linked to other moieties, for example a peptide.

Hybridization reactions can be sensitive and selective so that a particular sequence of interest can be identified even in samples in which it is present at low concentrations. In an in vitro situation, suitably stringent conditions can be defined by, for example, the concentrations of salt or formamide in the prehybridization and hybridization solutions, or by the hybridization temperature. In particular, stringency can be increased by reducing the concentration of salt, increasing the concentration of formamide, or raising the hybridization temperature. For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 mg/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are envisioned as well.

As such the methods and platforms described herein may utilize analysis of a nucleic acid molecule, such as sequencing a nucleic acid molecule. Sequencing methods may include whole genome sequencing, next generation sequencing, Sanger-sequencing, 16S rDNA sequencing and 16S rRNA sequencing. Further, such methods and platforms described herein may utilize mass-spectrometry, quantitative PCR, immunofluorescence, in situ hybridization, a microbial staining based platform, or combination thereof.

In some embodiments, the input to the identification platform can be any nucleic acid, including DNA, RNA, cDNA, miRNA, mtDNA, single or double-stranded. This nucleic acid can be of any length, as short as oligos of about 5 bp to as long as a megabase or even longer. As used herein, the term “nucleic acid molecule” means DNA, RNA, single-stranded, double-stranded or triple stranded and any chemical modifications thereof. Virtually any modification of the nucleic acid is contemplated. A “nucleic acid molecule” can be of almost any length, from 10, 20, 30, 40, 50, 60, 75, 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 1,500,000, 2,000,000, 5,000,000 or even more bases in length, up to a full-length chromosomal DNA molecule. For methods that analyze expression of a gene, the nucleic acid isolated from a sample is typically RNA.

Micro-RNAs (miRNA) are small single stranded RNA molecules an average of 22 nucleotides long that are involved in regulating mRNA expression in diverse species including humans (reviewed in Bartel 2004). The first report of miRNA was that of the lin-4 gene, discovered in the worm C. elegans (Lee, Feinbaum et al. 1993). Since then hundreds of miRNAs have been discovered in flies, plants and mammals. miRNAs regulate gene expression by binding to the 3′-untranslated regions of mRNA and catalyze either i) cleavage of the mRNA; or 2) repression of translation. The regulation of gene expression by miRNAs is central to many biological processes such as cell development, differentiation, communication, and apoptosis (Reinhart, Slack et al. 2000; Baehrecke 2003; Brennecke, Hipfner et al. 2003; Chen, Li et al. 2004). It has been shown that miRNA are active during embryogenesis of the mouse epithelium and play a significant role in skin morphogenesis (Yi, O'Carroll et al. 2006).

Given the role of miRNA in gene expression it is clear that miRNAs will influence, if not completely specify the relative amounts of mRNA in particular cell types and thus determine a particular gene expression profile (i.e., a population of specific mRNAs) in different cell types. In addition, it is likely that the particular distribution of specific miRNAs in a cell will also be distinctive in different cell types. Thus, determination of the miRNA profile of a tissue may be used as a tool for expression profiling of the actual mRNA population in that tissue. Accordingly, miRNA levels and/or detection of miRNA mutations are useful for the purposes of disease detection, diagnosis, prognosis, or treatment-related decisions (i.e., indicate response either before or after a treatment regimen has commenced) or characterization of a particular disease in the subject.

In embodiments, nucleic acid molecules can also be isolated by lysing the cells and cellular material collected from the skin sample by any number of means well known to those skilled in the art. For example, a number of commercial products available for isolating polynucleotides, including but not limited to, RNeasy™ (Qiagen, Valencia, Calif.) and TriReagent™ (Molecular Research Center, Inc, Cincinnati, Ohio) can be used. The isolated polynucleotides can then be tested or assayed for particular nucleic acid sequences, including a polynucleotide encoding a cytokine. Methods of recovering a target nucleic acid molecule within a nucleic acid sample are well known in the art, and can include microarray analysis.

As discussed further herein, nucleic acid molecules may be analyzed in any number of ways known in the art that may assist in determining the microbiome and/or metabolome associated with an individual's skin. For example, the presence of nucleic acid molecules can be detected by DNA-DNA or DNA-RNA hybridization or amplification using probes or fragments of the specific nucleic acid molecule. Nucleic acid amplification based assays involve the use of oligonucleotides or oligomers based on the nucleic acid sequences to detect transformants containing the specific DNA or RNA.

In another embodiment, antibodies that specifically bind the expression products of the nucleic acid molecules of microbiome and/or metabolome may be used to characterize the skin lesion of the subject. The antibodies may be used with or without modification, and may be labeled by joining them, either covalently or non-covalently, with a reporter molecule.

A wide variety of labels and conjugation techniques are known by those skilled in the art and may be used in various nucleic acid and amino acid assays. Means for producing labeled hybridization or PCR probes for detecting sequences include oligolabeling, nick translation, end-labeling or PCR amplification using a labeled nucleotide. Alternatively, the nucleic acid molecules, or any fragments thereof, may be cloned into a vector for the production of an mRNA probe. Such vectors are commercially available, and may be used to synthesize RNA probes in vitro by addition of an appropriate RNA polymerase such as T7, T3, or SP6 and labeled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio). Suitable reporter molecules or labels, which may be used for ease of detection, include radionuclides, enzymes, fluorescent, chemiluminescent, or chromogenic agents as well as substrates, cofactors, inhibitors, magnetic particles, and the like.

PCR systems usually use two amplification primers and an additional amplicon-specific, fluorogenic hybridization probe that specifically binds to a site within the amplicon. The probe can include one or more fluorescence label moieties. For example, the probe can be labeled with two fluorescent dyes: 1) a 6-carboxy-fluorescein (FAM), located at the 5′-end, which serves as reporter, and 2) a 6-carboxy-tetramethyl-rhodamine (TAMRA), located at the 3′-end, which serves as a quencher. When amplification occurs, the 5′-3′ exonuclease activity of the Taq DNA polymerase cleaves the reporter from the probe during the extension phase, thus releasing it from the quencher. The resulting increase in fluorescence emission of the reporter dye is monitored during the PCR process and represents the number of DNA fragments generated. In situ PCR may be utilized for the direct localization and visualization of target nucleic acid molecules and may be further useful in correlating expression with histopathological finding.

Means for producing specific hybridization probes for nucleic acid molecules of the invention include the cloning of the nucleic acid sequences into vectors for the production of mRNA probes. Such vectors are commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as 32P or 35S, or enzymatic labels, such as alkaline phosphatase coupled to the probe via avidin/biotin coupling systems, and the like

P. acnes is a commensal, non-sporulating bacilliform (rod-shaped), gram-positive bacterium found in a variety of locations on the human body including the skin, mouth, urinary tract and areas of the large intestine. P. acnes can consume skin oil and produce byproducts such as short-chain fatty acids and propionic acid, which are known to help maintain a healthy skin barrier. Propionibacteria such as P. acnes also produce bacteriocins and bacteriocin-like compounds (e.g., propionicin P1G-1, jenseniin G, propionicins SM1, SM2 T1, and acnecin), which are inhibitory toward undesirable lactic acid-producing bacteria, gram-negative bacteria, yeasts, and molds. In some embodiments, a subject having skin identified as having P. acnes may be treated with a personal care product designed to inhibit growth and proliferation of P. acnes.

In some embodiments, an individual's skin profile is translated into a personalized SkinIQ™ index, which is an overall snapshot of skin health, by capturing both the diversity of skin flora and its eminence to assist in formulating a personal care product. The main factor contributing to eminence is probiotic balance, the ratio of mutualistic and commensal microorganisms to (opportunistic) pathogens. However eminence could also comprise other factors that could positively impact the health of skin. These factors could include presence of key biosynthetic microbial genes, gene products or proteins responsible for the promotion or maintenance of healthy host skin. All these factors will contribute to the collective health of skin by, but not limited to, the reduction of skin inflammation, the reduction of the relative amounts of pathogens, and the biosynthesis of pro-vitamins, antimicrobial peptides, vitamins and fatty acids. The combination of diversity and eminence, represented by SkinIQ™ index, can also be a predictive measure of skin health. For example, a preponderance of a certain subspecies of Propionibacterium acnes may be strongly associated with risk of acne breakout. Similarly, SkinIQ™ may be predictive of flare ups of other skin conditions including, but not limited to, eczema, psoriasis, atopic dermatitis and rosacea.

The SkinIQ™ index is defined under Skin Health Measurement System that contrasts any individual profile to the “consensus healthy profile” from a database of skin profiles (microbiomes and/or metabolomes) and places every profile within the healthy population context. The consensus healthy profile is defined separately for each bacterial species. The data from the healthy population is used to define the range where any given bacterial species is expected to be found within healthy individuals. All these ranges define a reference for future comparisons. The Skin Health Measurement System™ further serves as a powerful discovery tool that can be used to mine a rich data set for novel microbes that can be utilized in skin care formulations to positively impact different skin conditions including, but not limited to acne, atopic dermatitis, psoriasis and eczema. Also it can be used to mine higher-level interactions between different bacterial species, with potential therapeutic implications.

As such, the invention contemplates generating a reference database containing a number of reference projected profiles created from skin samples of subjects with known states, such as normal or healthy skin, as well as various skin disease states. The individuals profile may be compared with the reference database containing the reference profiles. If the profile of the subject matches best with the profile of a particular disease state in the database, the subject is diagnosed as having such disease state. Various computer systems and software can be utilized for implementing the analytical methods of this invention and are apparent to one of skill in the art. Exemplary software programs include, but are not limited to, Cluster & TreeView (Stanford, URLs: rana.lbl.gov or microarray.org), GeneCluster (MIT/Whitehead Institute, URL: MPR/GeneCluster/GeneCluster.html), Array Explorer (SpotFire Inc, URL: spotfire.com/products/scicomp.asp#SAE) and GeneSpring (Silicon Genetics Inc, URL: sigenetics.com/Products/GeneSpring/index.html) (for computer systems and software, see also U.S. Pat. No. 6,203,987, incorporated herein by reference).

In some embodiments, the invention provides a method of characterizing skin and/or subcutaneous tissue comprising collecting a sample from a subject containing skin or subcutaneous tissue flora. Skin and subcutaneous tissue flora of healthy individuals can be collected using swiping, scraping, swabbing, using tape strips or any other effective microbial collection method. The harvested sample can be profiled on a NGS, Sanger-sequencing, mass-spectrometry, quantitative PCR, immunofluorescence, in situ hybridization, or microbial staining based platform. For sequencing-based platforms, this can be done either using a whole-genome sequencing approach, or via targeted applications, a prominent example of which is 16S rDNA sequencing. All the above-mentioned identification methods can be implemented on samples directly collected from individuals without any proliferation step. This way, minimal bias is introduced toward identification of a mixture of culturable and unculturable microorganisms. A proprietary analysis algorithm can be used to identify species composition of each individual. A consensus healthy profile may be constructed from the healthy cohort. The healthy profile may be updated real time as more samples are collected over time. The healthy profile will serve as the reference for comparing all individual samples, i.e. profiles. Examples of identified bacteria belong to any phylum, including Actinobacteria, Firmicutes, Proteobacteria, Bacteroidetes. It will typically include common species such as Propionibacteria, Staphylococci, Corynebacteria, and Acenitobacteria species.

In some embodiments, the invention provides a platform or method for characterizing skin and subcutaneous tissue microbial flora of individuals with skin conditions. Skin and subcutaneous tissue flora of individuals with skin conditions that are considered to be suboptimal can be collected using swiping, swabbing, tape strips or any other effective microbial collection method. Collected microbial sample can be profiled on a NGS, Sanger-sequencing, mass-spectrometry, quantitative PCR, immunofluorescence, in situ hybridization, or microbial staining based platform. For the sequencing based platforms, this can be done either using a whole-genome sequencing approach, or via targeted applications, a prominent example of which is 16S rDNA sequencing. All the identification methods can be implemented on samples directly collected from individuals without any proliferation step. This way, minimal bias is introduced toward identification of a mixture of culturable and unculturable microorganisms. A personal skin and subcutaneous tissue flora profile can be generated for each individual. Individuals, based on their phenotypic characteristics, can be placed under specific skin condition categories as well. Such clustering effort will help to identify biological significant patterns which are characteristic of each cohort. The microbial composition of the affected cohort is distinct from the healthy profile. Microbial species which are associated with any given skin condition can be used as early diagnostic markers for individuals who have not developed a visual skin condition but may be prone to that. Examples of identified bacteria belong to any phylum, including Actinobacteria, Firmicutes, Proteobacteria, Bacteroidetes. It will typically include common species, such as Propionibacteria, Staphylococci, Corynebacteria, and Acenitobacteria species. Damaged skin can impact the composition of bacterial flora or can cause nonpathogenic bacteria to become pathogenic.

In some embodiments, the invention provides a platform or method for characterizing a consensus healthy skin and subcutaneous tissue metabolite profile. The metabolome associated with skin and subcutaneous tissue flora can also be profiled either by a mass-spectrometry based system or using genomics-based metabolome modeling and flux-balance analysis. Extraction can be done on samples collected by using swiping, swabbing, tape strips or any other effective microbial collection method. Alternatively, those metabolites and biochemical, specifically the extracellular ones, can be directly isolated from any individual without going through any cell harvesting. Characterization can be done on the whole metabolome or only be focused on a subset of metabolites, which are known or may be shown to be of significance in a particular disease pathology. All the above-mentioned identification methods can be implemented on samples directly collected from individuals without any proliferation step. This way, minimal bias is introduced in the population composition. A proprietary analysis algorithm may be used to identify metabolite composition of each individual's skin flora. A consensus healthy profile may be constructed from the healthy cohort. The healthy profile may be updated real time as more samples are collected over time. The healthy profile will serve as the reference for comparing all individual samples, i.e. profiles.

In some embodiments, the invention provides a platform or method for characterizing skin and subcutaneous tissue microbial flora of individuals with skin conditions. Metabolite composition of skin and subcutaneous tissue flora of individuals with skin conditions that are considered to be suboptimal can be profiled either by a mass-spectrometry based system or using genomics-based metabolome modeling and flux-balance analysis. Extraction can be done on samples collected by using swiping, swabbing, tape strips or any other effective microbial collection method. Alternatively, those metabolites and biochemical, specifically the extracellular ones, can be directly isolated from any individual without going through any cell harvesting. Characterization can be done on the whole metabolome or only be focused on a subset of metabolites, which are known or may be shown to be of significance. All the above-mentioned identification methods can be implemented on samples directly collected from individuals without any proliferation step. This way, minimal bias is introduced in the population composition. A personal profile can be generated for each individual that reflects the metabolite composition of the skin and subcutaneous tissue flora. Individuals, based on their phenotypic characteristics, can be placed under specific skin condition categories as well. Such clustering effort will help to identify biological significant patterns that are characteristic of each cohort. The metabolite composition of the affected cohort is distinct from the healthy profile. Metabolites which are associated with any given skin condition can be used as early diagnostic markers for individuals who have not developed a visual skin condition but may be prone to that.

### Systems and Methods for Providing Customized Treatments

Traditional treatments of dermatological conditions include use of antibiotics and/or anti-inflammatories. An unwanted side-effect of antibiotics (and especially antibiotics that have an overly broad spectrum) tend to alter an individual's microbiome in ways that are more detrimental than beneficial. That is, antibiotics are best suited for treating bacterial infections whereas many dermatologic disease processes are associated with or caused by bacterial overgrowth which creates bacterial/micro-organism imbalance. Infection differs from micro-organism imbalance in a number of ways. Fundamentally, infection is treated by eradication of the infectious micro-organism whereas micro-organism imbalance is typically best treated by adjusting or re-equilibrating the balance of micro-organism in areas of skin where the imbalance exists, and not by eradication of the micro-organism in the affected area. As such, antibiotic treatment of individuals who suffer certain dermatologic disorders tends to eradicate bacteria including normal components of the microbiome, rather than re-equilibrating the micro-organisms of the microbiome, and as such, antibiotic treatment tends to cause certain adverse effects and imbalances. Anti-inflammatory agents, and in particular steroid base anti-inflammatory agents, tend to attenuate the body's immune response and thus attenuating the epidermal cell's response to pathogens and as such tend to have certain adverse effects. Along the same lines, traditional antibiotic and anti-inflammatory treatments tend not to address the underlying pathophysiology of the certain dermatologic disorders, because, for example, these traditional therapies tend to be overly broad in their mode of action which results in many cases in harmful imbalances in the microbiome and immune system of the individual being treated.

Described herein are customized skin care and personal care products for human and animal use and, more particularly, but not by way of limitation, the development of personal care products that are based on the initial evaluation of the flora inhabiting the skin and subcutaneous tissue. Described herein are systems and methods for analyzing the skin and subcutaneous tissue flora and its associated metabolome, comparing the resulting profile of the skin and subcutaneous tissue flora and metabolome to a healthy profile, represented as a quantity and diversity of flora that falls within a range determined from a set of healthy skin types, and then customizing skin care and personal care products that will augment the flora residing on a test subject's skin and subcutaneous tissue and its associated metabolome or replicate a healthy flora profile on to that of a test subject.

Individualized skin test result are used as the basis for development of individualized skin care and personal care products which are customized to either maintain a healthy skin microbiome and metabolome or shift a profile towards a healthy equilibrium or state by adding one or more commensal and/or mutualistic organisms and/or substrates that favor the growth of commensal and mutualistic organisms on the skin.

The exact composition of the skin care product blend may be determined after comparing the resulting profile of any individual's skin and subcutaneous tissue flora and metabolome to a healthy profile and then customizing skin care and personal care products that best shift the subject's skin and subcutaneous tissue flora and metabolome toward a healthy profile. The optimal flora and substrates and metabolomes would also synergize with host's immune system and contribute toward a healthy skin from that perspective.

Furthermore, the composition of subject's flora and metabolome may be compared to previously complied database of different skin conditions to see whether he or she is prone to develop any of those skin conditions in future. Based on the customized or personalized test results, a customized or personalized skin care or personal care blend may be formulated for that individual by blending a mixture of commensal and mutualistic microorganisms or their relevant metabolites that are depleted in that individual's flora or metabolome with or without the necessary substrates and nutrients that favor proliferation of commensal and mutualistic organisms. This customized or personalized skin care or personal care product is specifically created in a way to establish an optimal profile by either maintaining a healthy microbiome or shifting the suboptimal profile towards a healthy equilibrium. Also the synergies between the optimal microbial flora and its associated metabolome and host's immune system will further contribute to skin health and wellness.

Skin care products or personal care products suitable for use with the systems and methods described herein, in some embodiments, include skin care products and include, but are not limited to, cleansing products, shampoo, conditioner, toners or creams, topical ointments and gels, as well as localized (e.g. under eye) gel, all of which may be formulated to contain ingredients specifically designed to shift microbial population to a healthy profile with or without a commensal or mutualistic organism or mixture of commensal or mutualistic organisms in either an active or dormant state. Such skin care products may further include therapeutic agents, vitamins, antioxidants, minerals, skin toning agents, polymers, excipients, surfactants, probiotics or fraction thereof, microorganism or product from the culture thereof, such a bacteria, fungi and the like, either living, dormant or inactive.

In some embodiments, the platform or method described herein may be provided as a test for profiling the skin flora of any individual, either healthy or with a skin condition and also their associated metabolome. Such test would be sensitive to characterize the dominant skin flora and metabolites of any individual. A customized or personalized evaluation of any individual's flora may be conducted and identified skin and subcutaneous tissue flora and metabolites may be compared to healthy and also affected skin profiles. A customized or personalized report may be generated which will specify species composition of the individual's skin and subcutaneous tissue flora and also its associated metabolites. Such report will enlist the beneficial and commensal species that are depleted or over-represented in each individual. It will also include the list of beneficial or undesired metabolites that are either depleted or over-represented in each individual. This may be used for formulation of the customized or personalized skin care or personal care product. Alternatively, the test can be administered to assess the performance of other skin care and personal care products, therapies, or evaluate any disruption of the normal skin flora or metabolites. The test can be performed before, during, and after any skin treatment in order to monitor the efficacy of that treatment regimen on skin flora or its associated metabolites. The test can also be used for early diagnostic of skin conditions that are associated with a signature microbial profile or their accompanying metabolites. The sensitivity of the test allows early diagnostic of such skin conditions before their phenotypic outbreak. In an aspect, the invention provides a method for generating, or a customized or personalized skin care or personal care product formulated for a particular individual. The customized or personalized product contains one or more beneficial or commensal microorganisms or a set of chemicals and metabolites which may be depleted in any given individual. Regular administration of such skin care products and personal care products should shift the suboptimal profile towards a healthy equilibrium. Skin care product may be applied after cleansing the existing flora with a proprietary lotion that will enhance the efficacy of colonization of skin care product microorganisms or its constituent metabolites. Any customized or personalized skin care or personal care product can contain one or more microorganisms, culturable or unculturable. The customized or personalized product can alternatively be a substrate and nutrients that favor the establishment or proliferation of mutualistic or commensal organisms and/or suppression of pathogenic organisms. Those chemicals and metabolites are either synthesized in vitro or purified from a microorganism.

### Metabolic Modelling

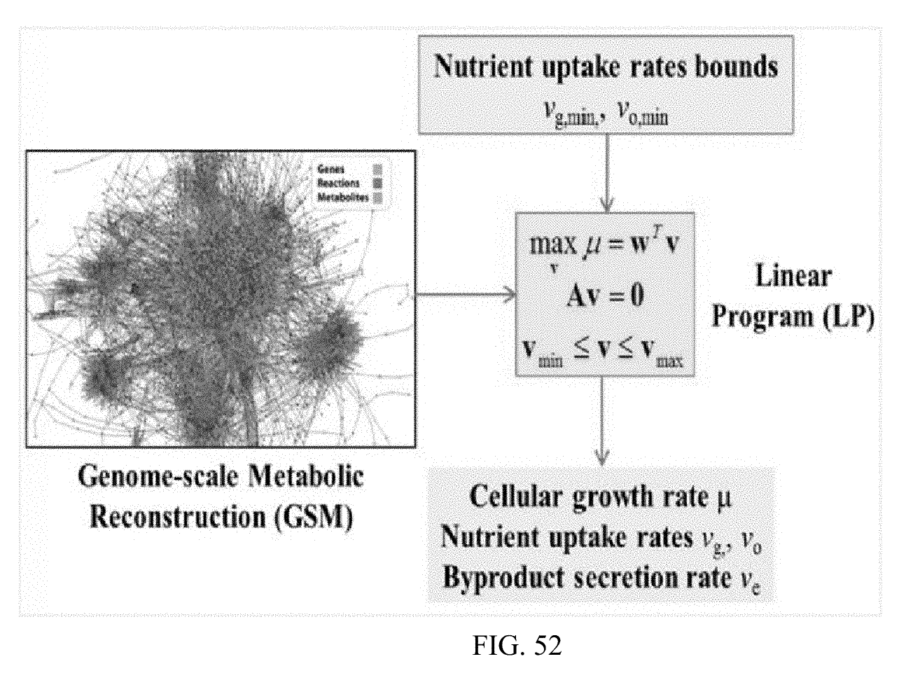

FIG. 52 shows a schematic representation of an algorithm for modeling a Genome-Scale Metabolic reconstruction or GSM as used in embodiments of the systems and methods described herein. In some embodiments, a machine learning algorithm receives data extracted from samples comprising the microbiome and metabolome data associated with a particular sample such as a skin or hair sample. A machine learning algorithm is first trained to generate a reference database comprising threshold values for various micro-organisms and micro-organism metabolites associated with samples taken from known disease free and/or disease having individuals. The machine learning algorithm models this reference data with respect to such factors as, for example, nutrient uptake rate, cellular growth rate, and byproduct secretion rate. The machine learning algorithm receives new sample data comprising microbiome and/or metabolome data and compares new sample data against the threshold values to determine a characterization of the sample. Table 1 below shows exemplary data used to train an embodiment of the machine learning algorithm with respect to microbiomes and metabolomes of healthy individuals from the skin and/or hair samples taken from these individuals.

TABLE 1

| | |
|---|---|
| FORMULA: | C20H21N7O7 |
| BioCyc: | META:10-FORMYL-THF |
| SEED Compound: | cpd00201 |
| UniPathway Compound: | UPC00234 |
| KEGG Compound: | C00234 |
| BioPath Molecule: | 10-Formyl-5,6,7,8-tetrahydrofolate |
| MetaNetX (MNX) Chemical: | MNXM237 |
| Reactome: | 419151; 5389850 |
| Human Metabolome Database: | HMDB00972 |
| FORMULA: | C10H12N5O10P2 |
| BioCyc: | META:ADP; META:ADP-GROUP |
| SEED Compound: | cpd00008 |
| UniPathway Compound: | UPC00008 |
| KEGG Compound: | C00008; G11113 |
| BioPath Molecule: | Adenosine-5-prime-diphosphate |
| MetaNetX (MNX) Chemical: | MNXM7 |
| Reactome: | 113581; 113582; 114565; 211606; |
| | 29370; 5632457 |
| Human Metabolome Database: | HMDB01341 |
| FORMULA: | C10H12N5O13P3 |
| BioCyc: | META:ATP |
| SEED Compound: | cpd00002 |
| UniPathway Compound: | UPC00002 |
| KEGG Compound: | C00002; D08646 |
| BioPath Molecule: | Adenosine-5-prime-triphosphate |
| MetaNetX (MNX) Chemical: | MNXM3 |
| Reactome: | 211579; 389573 |
| Human Metabolome Database: | HMDB00538 |

In some embodiments, clinical tests measuring the efficacy of benzoyl peroxide for the treatment of acnes vulgaris are utilized to build a machine learning model. In some embodiments, the machine learning model os built to predict whether an individual will respond positively to benzoyl peroxide or not with an accuracy of at least 60%, at least 65%, at least 70%, at least 75%, at least 80%, at least 85%, at least 90%, or at least 95%. In some embodiments, the accuracy of the machine learning model is about 60%, about 65%, about 70%, about 75%, about 80%, about 85%, about 90%, or about 95%. In some embodiments, the positive predictive value of the machine learning model is at least 60%, at least 65%, at least 70%, at least 75%, at least 80%, at least 85%, at least 90%, or at least 95%. In some embodiments, the positive predictive value of the machine learning model is about 60%, about 65%, about 70%, about 75%, about 80%, about 85%, about 90%, or about 95%. In some embodiments, the negative predictive value of the machine learning model is at least 60%, at least 65%, at least 70%, at least 75%, at least 80%, at least 85%, at least 90%, or at least 95%. In some embodiments, the negative predictive value of the machine learning model is about 60%, about 65%, about 70%, about 75%, about 80%, about 85%, about 90%, or about 95%.

### Machine Learning Algorithms

Described herein are devices, software, systems, and methods that apply one or more algorithms for analyzing input data to generate predictions relating treating skin conditions with medicaments. In some embodiments, the skin condition is acne vulgaris and the medicament is one or more anti-acne medication. In some embodiments, the algorithms utilize statistical modeling to generate predictions or estimates about the effects of one or more medicament on a subject's skin microbiome. In some embodiments, machine learning algorithms are used for training prediction models and/or making predictions. In some embodiments, the algorithm predicts a likelihood or probability of one or more properties or functions. In some embodiments, an algorithm utilizes a predictive model such as a neural network, a decision tree, a support vector machine, or other applicable model. Using the training data, an algorithm is able to form a classifier for generating a classification or prediction according to relevant features. The features selected for classification can be classified using a variety of methods. In some embodiments, the trained algorithm comprises a machine learning algorithm.

In some embodiments, the machine learning algorithm uses a support vector machine (SVM), a Naïve Bayes classification, a random forest, or an artificial neural network. Machine learning techniques include bagging procedures, boosting procedures, random forest algorithms, and combinations thereof. In some embodiments, the predictive model is a deep neural network. In some embodiments, the predictive model is a deep convolutional neural network.

In some embodiments, a machine learning algorithm uses a supervised learning approach. In supervised learning, the algorithm generates a function from labeled training data. Each training example is a pair consisting of an input object and a desired output value. In some embodiments, an optimal scenario allows for the algorithm to correctly determine the class labels for unseen instances. In some embodiments, a supervised learning algorithm requires the user to determine one or more control parameters. These parameters are optionally adjusted by optimizing performance on a subset, called a validation set, of the training set. After parameter adjustment and learning, the performance of the resulting function is optionally measured on a test set that is separate from the training set. Regression methods are commonly used in supervised learning. Accordingly, supervised learning allows for a model or classifier to be generated or trained with training data in which the expected output is known in advance.

In some embodiments, a machine learning algorithm uses an unsupervised learning approach. In unsupervised learning, the algorithm generates a function to describe hidden structures from unlabeled data (e.g., a classification or categorization is not included in the observations). Since the examples given to the learner are unlabeled, there is no evaluation of the accuracy of the structure that is output by the relevant algorithm. Approaches to unsupervised learning include: clustering, anomaly detection, and approaches based on neural networks including autoencoders and variational autoencoders.

In some embodiments, the machine learning algorithm utilizes multi-class learning. Multi-task learning (MTL) is an area of machine learning in which more than one learning task is solved simultaneously in a manner that takes advantage of commonalities and differences across the multiple tasks. Advantages of this approach can include improved learning efficiency and prediction accuracy for the specific predictive models in comparison to training those models separately. Regularization to prevent overfitting can be provided by requiring an algorithm to perform well on a related task. This approach can be better than regularization that applies an equal penalty to all complexity. Multi-class learning can be especially useful when applied to tasks or predictions that share significant commonalities and/or are under-sampled. In some embodiments, multi-class learning is effective for tasks that do not share significant commonalities (e.g., unrelated tasks or classifications). In some embodiments, multi-class learning is used in combination with transfer learning.

In some embodiments, a machine learning algorithm learns in batches based on the training dataset and other inputs for that batch. In other embodiments, the machine learning algorithm performs additional learning where the weights and error calculations are updated, for example, using new or updated training data. In some embodiments, the machine learning algorithm updates the prediction model based on new or updated data. For example, a machine learning algorithm can be applied to new or updated data to be re-trained or optimized to generate a new prediction model. In some embodiments, a machine learning algorithm or model is re-trained periodically as additional data becomes available.

In some embodiments, the classifier or trained algorithm of the present disclosure comprises one feature space. In some cases, the classifier comprises two or more feature spaces. In some embodiments, the two or more feature spaces are distinct from one another. In some embodiments, the accuracy of the classification or prediction is improved by combining two or more feature spaces in a classifier instead of using a single feature space. The attributes generally make up the input features of the feature space and are labeled to indicate the classification of each case for the given set of input features corresponding to that case.

The accuracy of the classification may be improved by combining two or more feature spaces in a predictive model or classifier instead of using a single feature space. In some embodiments, the predictive model comprises at least two, three, four, five, six, seven, eight, nine, or ten or more feature spaces. The training data is fed into the machine learning algorithm which processes the input features and associated outcomes to generate a trained model or predictor. In some cases, the machine learning algorithm is provided with training data that includes the classification, thus enabling the algorithm to “learn” by comparing its output with the actual output to modify and improve the model. This is often referred to as supervised learning. Alternatively, in some instances, the machine learning algorithm is provided with unlabeled or unclassified data, which leaves the algorithm to identify hidden structure amongst the cases (e.g., clustering). This is referred to as unsupervised learning.

In some embodiments, one or more sets of training data are used to train a model using a machine learning algorithm. In some embodiments, the methods described herein comprise training a model using a training data set. Although exemplar embodiments of the present disclosure include machine learning algorithms that use deep neural networks, various types of algorithms are contemplated. In some embodiments, the algorithm utilizes a predictive model such as a neural network, a decision tree, a support vector machine, or other applicable model. In some embodiments, the machine learning algorithm is selected from the group consisting of a supervised, semi-supervised and unsupervised learning, such as, for example, a support vector machine (SVM), a Naïve Bayes classification, a random forest, an artificial neural network, a decision tree, a K-means, learning vector quantization (LVQ), self-organizing map (SOM), graphical model, regression algorithm (e.g., linear, logistic, multivariate, association rule learning, deep learning, dimensionality reduction and ensemble selection algorithms. In some embodiments, the machine learning algorithm is selected from the group consisting of: a support vector machine (SVM), a Naïve Bayes classification, a random forest, and an artificial neural network. Machine learning techniques include bagging procedures, boosting procedures, random forest algorithms, and combinations thereof. Illustrative algorithms for analyzing the data include but are not limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis.

### Computing Systems and Software

Described herein are devices, software, systems, and methods that apply one or more algorithms for analyzing input data to generate predictions relating to the treatment of skin conditions with one or more medicaments. In some embodiments, a system as described herein comprises a computing device such as a digital processing device. In some embodiments, a system as described herein comprises a network element for communicating with a server. In some embodiments, a system as described herein comprises a server. In some embodiments, the system is configured to upload to and/or download data from the server. In some embodiments, the server is configured to store input data, output, and/or other information. In some embodiments, the server is configured to backup data from the system or apparatus.

In some embodiments, the system comprises one or more digital processing devices. In some embodiments, the system comprises a plurality of processing units configured to generate the trained model(s). In some embodiments, the system comprises a plurality of graphic processing units (GPUs), which are amenable to machine learning applications. For example, GPUs are generally characterized by an increased number of smaller logical cores composed of arithmetic logic units (ALUs), control units, and memory caches when compared to central processing units (CPUs). Accordingly, GPUs are configured to process a greater number of simpler and identical computations in parallel, which are amenable to the math matrix calculations common in machine learning approaches. In some embodiments, the system comprises one or more tensor processing units (TPUs), which are AI application-specific integrated circuits (ASIC) developed by Google for neural network machine learning. In some embodiments, the methods described herein are implemented on systems comprising a plurality of GPUs and/or TPUs. In some embodiments, the systems comprise at least 2, 3, 4, 5, 6, 7, 8, 9, 10, 15, 20, 30, 40, 50, 60, 70, 80, 90, or 100 or more GPUs or TPUs. In some embodiments, the GPUs or TPUs are configured to provide parallel processing.

In some embodiments, the system or apparatus is configured to encrypt data. In some embodiments, data on the server is encrypted. In some embodiments, the system or apparatus comprises a data storage unit or memory for storing data. In some embodiments, data encryption is carried out using Advanced Encryption Standard (AES). In some embodiments, data encryption is carried out using 128-bit, 192-bit, or 256-bit AES encryption. In some embodiments, data encryption comprises full-disk encryption of the data storage unit. In some embodiments, data encryption comprises virtual disk encryption. In some embodiments, data encryption comprises file encryption. In some embodiments, data that is transmitted or otherwise communicated between the system or apparatus and other devices or servers is encrypted during transit. In some embodiments, wireless communications between the system or apparatus and other devices or servers is encrypted. In some embodiments, data in transit is encrypted using a Secure Sockets Layer (SSL).

An apparatus as described herein comprises a digital processing device that includes one or more hardware central processing units (CPUs) or general purpose graphics processing units (GPGPUs) that carry out the device's functions. The digital processing device further comprises an operating system configured to perform executable instructions. The digital processing device is optionally connected to a computer network. The digital processing device is optionally connected to the Internet such that it accesses the World Wide Web. The digital processing device is optionally connected to a cloud computing infrastructure. Suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein.

Typically, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing.

A digital processing device as described herein either includes or is operatively coupled to a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, a system or method as described herein generates a database as containing or comprising input and/or output data. Some embodiments of the systems described herein are computer based systems. These embodiments include a CPU including a processor and memory which may be in the form of a non-transitory computer readable storage medium. These system embodiments further include software that is typically stored in memory (such as in the form of a non-transitory computer readable storage medium) where the software is configured to cause the processor to carry out a function. Software embodiments incorporated into the systems described herein contain one or more modules.

In various embodiments, an apparatus comprises a computing device or component such as a digital processing device. In some of the embodiments described herein, a digital processing device includes a display to display visual information. Non-limiting examples of displays suitable for use with the systems and methods described herein include a liquid crystal display (LCD), a thin film transistor liquid crystal display (TFT-LCD), an organic light emitting diode (OLED) display, an OLED display, an active-matrix OLED (AMOLED) display, or a plasma display.

A digital processing device, in some of the embodiments described herein includes an input device to receive information. Non-limiting examples of input devices suitable for use with the systems and methods described herein include a keyboard, a mouse, trackball, track pad, or stylus. In some embodiments, the input device is a touch screen or a multi-touch screen.

The systems and methods described herein typically include one or more non-transitory computer readable storage media encoded with a program including instructions executable by the operating system of an optionally networked digital processing device. In some embodiments of the systems and methods described herein, the non-transitory storage medium is a component of a digital processing device that is a component of a system or is utilized in a method. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

Typically the systems and methods described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages. The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

Typically, the systems and methods described herein include and/or utilize one or more databases. In view of the disclosure provided herein, those of skill in the art will recognize that many databases are suitable for storage and retrieval of baseline datasets, files, file systems, objects, systems of objects, as well as data structures and other types of information described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

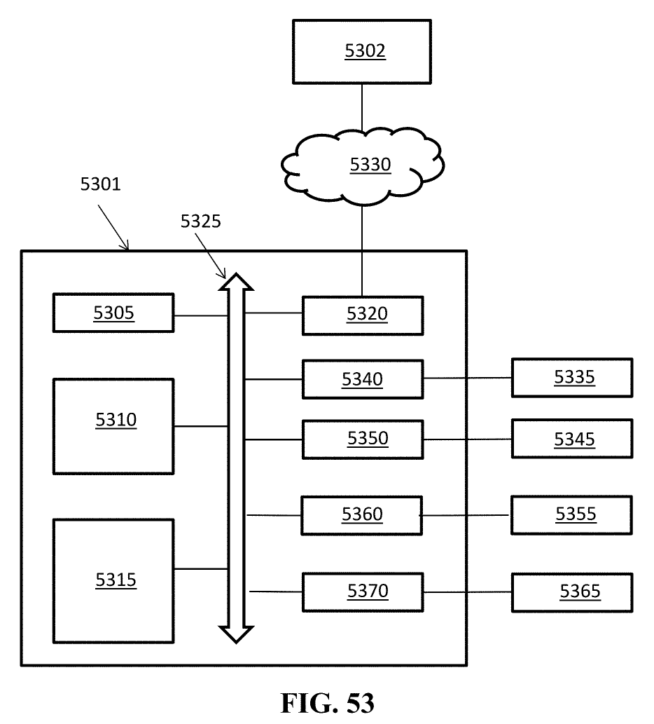

FIG. 53 shows an exemplary embodiment of a system as described herein comprising an apparatus such as a digital processing device 5301. The digital processing device 5301 includes a software application configured to analyze input data. The digital processing device 5301 may include a central processing unit (CPU, also “processor” and “computer processor” herein) 5305, which can be a single core or multi-core processor, or a plurality of processors for parallel processing. The digital processing device 5301 also includes either memory or a memory location 5310 (e.g., random-access memory, read-only memory, flash memory), electronic storage unit 5315 (e.g., hard disk), communication interface 5320 (e.g., network adapter, network interface) for communicating with one or more other systems, and peripheral devices, such as cache. The peripheral devices can include storage device(s) or storage medium 5365 which communicate with the rest of the device via a storage interface 5370. The memory 5310, storage unit 5315, interface 5320 and peripheral devices are configured to communicate with the CPU 5305 through a communication bus 5325, such as a motherboard. The digital processing device 5301 can be operatively coupled to a computer network (“network”) 5330 with the aid of the communication interface 5320. The network 5330 can comprise the Internet. The network 5330 can be a telecommunication and/or data network.

The digital processing device 5301 includes input device(s) 5345 to receive information, the input device(s) in communication with other elements of the device via an input interface 5350. The digital processing device 5301 can include output device(s) 5355 that communicates to other elements of the device via an output interface 5360.

The CPU 5305 is configured to execute machine-readable instructions embodied in a software application or module. The instructions may be stored in a memory location, such as the memory 5310. The memory 5310 may include various components (e.g., machine readable media) including, but not limited to, a random access memory component (e.g., RAM) (e.g., a static RAM “SRAM”, a dynamic RAM “DRAM, etc.), or a read-only component (e.g., ROM). The memory 5310 can also include a basic input/output system (BIOS), including basic routines that help to transfer information between elements within the digital processing device, such as during device start-up, may be stored in the memory 5310.

The storage unit 5315 can be configured to store files, such as medical data. The storage unit 5315 can also be used to store operating system, application programs, and the like. Optionally, storage unit 5315 may be removably interfaced with the digital processing device (e.g., via an external port connector (not shown)) and/or via a storage unit interface. Software may reside, completely or partially, within a computer-readable storage medium within or outside of the storage unit 5315. In another example, software may reside, completely or partially, within processor(s) 5305.

Information and data can be displayed to a user through a display 5335. The display is connected to the bus 5325 via an interface 5340, and transport of data between the display other elements of the device 5301 can be controlled via the interface 5340.

Methods as described herein can be implemented by way of machine (e.g., computer processor) executable code stored on an electronic storage location of the digital processing device 5301, such as, for example, on the memory 5310 or electronic storage unit 5315. The machine executable or machine readable code can be provided in the form of a software application or software module. During use, the code can be executed by the processor 5305. In some cases, the code can be retrieved from the storage unit 5315 and stored on the memory 5310 for ready access by the processor 5305. In some situations, the electronic storage unit 5315 can be precluded, and machine-executable instructions are stored on memory 5310.

In some embodiments, a remote device 5302 is configured to communicate with the digital processing device 5301, and may comprise any mobile computing device, non-limiting examples of which include a tablet computer, laptop computer, smartphone, or smartwatch. For example, in some embodiments, the remote device 5302 is a smartphone of the user that is configured to receive information from the digital processing device 5301 of the apparatus or system described herein in which the information can include a summary, input, output, or other data. In some embodiments, the remote device 5302 is a server on the network configured to send and/or receive data from the apparatus or system described herein. Some embodiments of the systems and methods described herein are configured to generate a database containing or comprising input and/or output data. A database, as described herein, is configured to function as, for example, a data repository for input and output data. In some embodiments, the database is stored on a server on the network. In some embodiments the database is stored locally on the apparatus (e.g., the monitor component of the apparatus). In some embodiments, the database is stored locally with data backup provided by a server.

### Methods of Treatment

Provided herein, in some aspects, are compositions for the treatment of acne, eczema, psoriasis, seborrheic dermatitis, rosacea, or any combination thereof. In some aspects, the disclosure provides methods for treating or preventing acne. In some embodiments, methods disclosed herein comprise administering a pharmaceutical composition comprising one or more anti-acne medication. In some embodiments, methods disclosed herein comprise administering daily. In some embodiments, methods disclosed herein comprise administering once daily. In some embodiments, methods disclosed herein comprise administering more than once daily. In some embodiments, methods disclosed herein comprise administering twice daily. In some embodiments, methods disclosed herein comprise administering three times daily. In some embodiments, methods disclosed herein comprise administering four times daily. In some embodiments, methods disclosed herein comprise administering once to three times daily. In some embodiments, methods disclosed herein comprise administering two to three times daily. In some embodiments, methods disclosed herein comprise administering two to four times daily. In some embodiments, methods disclosed herein comprise administering weekly. In some embodiments, methods disclosed herein comprise administering twice weekly. In some embodiments, methods disclosed herein comprise administering three times weekly. In some embodiments, methods disclosed herein comprise administering four times weekly. In some embodiments, methods disclosed herein comprise administering five times weekly. In some embodiments, methods disclosed herein comprise administering six times weekly. In some embodiments, methods disclosed herein comprise administering monthly.

In some embodiments, the methods, compositions, and systems described herein are effective at decreasing the population of one or more target bacterial genus, species, or strain on a subject's skin. In some embodiments, a target population is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population of one or more target bacterial genus, species, or strain on a subject's skin. In some embodiments, a target population is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, the methods, compositions, and systems described herein are effective at decreasing a population of P. acnes on a subject's skin. In some embodiments, a target population of P. acnes is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of P. acnes is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population P. acnes on a subject's skin. In some embodiments, a target population of P. acnes is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of P. acnes is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of P. acnes on a subject's skin.

In some embodiments, the methods, compositions, and systems described herein are effective at decreasing a population of S. mitis on a subject's skin. In some embodiments, a target population of S. mitis is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. mitis is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population S. mitis on a subject's skin. In some embodiments, a target population of S. mitis is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. mitis is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of S. mitis on a subject's skin.

In some embodiments, the methods, compositions, and systems described herein are effective at decreasing a population of R. mucilaginosa on a subject's skin. In some embodiments, a target population of R. mucilaginosa is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of R. mucilaginosa is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population R. mucilaginosa on a subject's skin. In some embodiments, a target population of R. mucilaginosa is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of R. mucilaginosa is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of R. mucilaginosa on a subject's skin.

In some embodiments, the methods, compositions, and systems described herein are effective at decreasing a population of S. sanguinis on a subject's skin. In some embodiments, a target population of S. sanguinis is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. sanguinis is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population S. sanguinis on a subject's skin. In some embodiments, a target population of S. sanguinis is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. sanguinis is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of S. sanguinis on a subject's skin.

In some embodiments, the methods, compositions, and systems described herein are effective at decreasing a population of H. parainfluenzae on a subject's skin. In some embodiments, a target population of H. parainfluenzae is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of H. parainfluenzae is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population H. parainfluenzae on a subject's skin. In some embodiments, a target population of H. parainfluenzae is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of H. parainfluenzae is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of H. parainfluenzae on a subject's skin.

In some embodiments, the methods, compositions, and systems described herein are effective at decreasing a population of S. pneumoniae on a subject's skin. In some embodiments, a target population of S. pneumoniae is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. pneumoniae is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population S. pneumoniae on a subject's skin. In some embodiments, a target population of S. pneumoniae is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. pneumoniae is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of S. pneumoniae on a subject's skin.

In some embodiments, the methods, compositions, and systems described herein are effective at decreasing a population of S. thermophilus on a subject's skin. In some embodiments, a target population of S. thermophilus is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. thermophilus is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population S. thermophilus on a subject's skin. In some embodiments, a target population of S. thermophilus is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. thermophilus is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of S. thermophilus on a subject's skin.

In some embodiments, the methods, compositions, and systems described herein are effective at decreasing a population of G. elegans on a subject's skin. In some embodiments, a target population of G. elegans is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of G. elegans is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population G. elegans on a subject's skin. In some embodiments, a target population of G. elegans is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of G. elegans is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of G. elegans on a subject's skin.

In some embodiments, the methods, compositions, and systems described herein are effective at decreasing a population of G. haemolysans on a subject's skin. In some embodiments, a target population of G. haemolysans is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of G. haemolysans is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population G. haemolysans on a subject's skin. In some embodiments, a target population of G. haemolysans is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of G. haemolysans is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of G. haemolysans on a subject's skin.

In some embodiments, the methods, compositions, and systems described herein are effective at decreasing a population of S. oralis on a subject's skin. In some embodiments, a target population of S. oralis is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. oralis is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population S. oralis on a subject's skin. In some embodiments, a target population of S. oralis is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. oralis is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of S. oralis on a subject's skin.

In some embodiments, the methods, compositions, and systems described herein are effective at decreasing a population of S. epidermis on a subject's skin. In some embodiments, a target population of S. epidermis is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. epidermis is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population S. epidermis on a subject's skin. In some embodiments, a target population of S. epidermis is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. epidermis is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of S. epidermis on a subject's skin.

In some embodiments, the methods, compositions, and systems described herein are effective at decreasing a population of V. parvula on a subject's skin. In some embodiments, a target population of V. parvula is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of V. parvula is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population V. parvula on a subject's skin. In some embodiments, a target population of V. parvula is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of V. parvula is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of V. parvula on a subject's skin.

In some embodiments, the methods, compositions, and systems described herein are effective at decreasing a population of S. goronii on a subject's skin. In some embodiments, a target population of S. goronii is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. goronii is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population S. goronii on a subject's skin. In some embodiments, a target population of S. goronii is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of S. goronii is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of S. goronii on a subject's skin.

In some embodiments, the methods, compositions, and systems described herein are effective at decreasing a population of N. macacae on a subject's skin. In some embodiments, a target population of N. macacae is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of N. macacae is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods, compositions, and systems described herein are effective at increasing the population N. macacae on a subject's skin. In some embodiments, a target population of N. macacae is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, a target population of N. macacae is reduced by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the application of benzoyl peroxide is effective at increasing or decreasing a population of N. macacae on a subject's skin.

### Pharmaceutical Compositions

Provided herein, in some aspects, are compositions that comprise at least one or more anti-acne medication disclosed herein, wherein the compositions are formulated for administration to a subject in need thereof. Generally, the subject is a human afflicted with acne, eczema, psoriasis, seborrheic dermatitis, rosacea, or any combination thereof. In some embodiments, a composition is formulated for topical administration to a subject in need thereof. In some embodiments, the compositions are formulated for topical administration to the skin of the subject. In some embodiments, a composition is formulated for transdermal administration. In certain embodiments, the composition is a formulation selected from a gel, ointment, lotion, emulsion, paste, cream, foam, mousse, liquid, spray, suspension, dispersion and aerosol. In certain embodiments, the formulation comprises one or more excipients to provide a desired form and a desired viscosity, flow or other physical or chemical characteristic for effective application, coverage and adhesion to skin.

Compositions disclosed herein may be presented in a formulation that includes one or more excipients to improve any one or more of shelf-life, application, and therapeutic effect. In some embodiments, the excipient is necessary to improve any one or more of shelf-life, application, skin penetration, and therapeutic effect. In some embodiments, the excipient reduces skin penetration.

Compositions disclosed herein may be presented in a formulation that includes one or more stabilizers to improve any one or more of shelf-life, storage viability, and freeze thawing survival. In some embodiments, examples of stabilizers include, but are not limited to, glyconanoparticle, a liposome, a nanoparticle, trehalose, sucrose, stachyose, hydroxyethyl starch, glycine, mannitol, DMSO, ethylene glycol, propylene glycol (PG), polypropylene glycol (PPG), polyethylene glycol (PEG), and 2-Methyl-2,4-pentanediol (MPD). In some embodiments, the stabilizer eliminates a need for temperature control, e.g., cold chain storage.

In certain embodiments, the compositions and formulations described herein comprise one or more anti-acne medication at a concentration of between about 0.01% w/v and about 99% w/v, about 0.1% w/v and about 99% w/v, about 1% w/v and about 99% w/v, between about 1% w/v to about 90% w/v, between about 1% w/v to about 5% w/v, between about 1% w/v to about 10% w/v, between about 1% w/v to about 15% w/v, between about 1% w/v to about 20% w/v, between about 1% w/v to about 30% w/v, between about 1% w/v to about 40% w/v, between about 1% w/v to about 50% w/v, between about 1% w/v to about 60% w/v, between about 1% w/v to about 70% w/v, between about 1% w/v to about 80% w/v, between about 1% w/v to about 90% w/v, between about 5% w/v to about 10% w/v, between about 5% w/v to about 15% w/v, between about 5% w/v to about 20% w/v, between about 5% w/v to about 30% w/v, between about 5% w/v to about 40% w/v, between about 5% w/v to about 50% w/v, between about 5% w/v to about 60% w/v, between about 5% w/v to about 70% w/v, between about 5% w/v to about 80% w/v, between about 5% w/v to about 90% w/v, between about 10% w/v to about 15% w/v, between about 10% w/v to about 20% w/v, between about 10% w/v to about 30% w/v, between about 10% w/v to about 40% w/v, between about 10% w/v to about 50% w/v, between about 10% w/v to about 60% w/v, between about 10% w/v to about 70% w/v, between about 10% w/v to about 80% w/v, between about 10% w/v to about 90% w/v, between about 15% w/v to about 20% w/v, between about 15% w/v to about 30% w/v, between about 15% w/v to about 40% w/v, between about 15% w/v to about 50% w/v, between about 15% w/v to about 60% w/v, between about 15% w/v to about 70% w/v, between about 15% w/v to about 80% w/v, between about 15% w/v to about 90% w/v, between about 20% w/v to about 30% w/v, between about 20% w/v to about 40% w/v, between about 20% w/v to about 50% w/v, between about 20% w/v to about 60% w/v, between about 20% w/v to about 70% w/v, between about 20% w/v to about 80% w/v, between about 20% w/v to about 90% w/v, between about 30% w/v to about 40% w/v, between about 30% w/v to about 50% w/v, between about 30% w/v to about 60% w/v, between about 30% w/v to about 70% w/v, between about 30% w/v to about 80% w/v, between about 30% w/v to about 90% w/v, between about 40% w/v to about 50% w/v, between about 40% w/v to about 60% w/v, between about 40% w/v to about 70% w/v, between about 40% w/v to about 80% w/v, between about 40% w/v to about 90% w/v, between about 50% w/v to about 60% w/v, between about 50% w/v to about 70% w/v, between about 50% w/v to about 80% w/v, between about 50% w/v to about 90% w/v, between about 60% w/v to about 70% w/v, between about 60% w/v to about 80% w/v, between about 60% w/v to about 90% w/v, between about 70% w/v to about 80% w/v, between about 70% w/v to about 90% w/v, about 80% w/v to about 90% w/v, between about 0.01% w/v to about 5% w/v, between about 0.01% w/v to about 0.05% w/v, between about 0.01% w/v to about 0.1% w/v, between about 0.01% w/v to about 0.5% w/v, between about 0.01% w/v to about 1% w/v, between about 0.01% w/v to about 2% w/v, between about 0.01% w/v to about 3% w/v, between about 0.01% w/v to about 4% w/v, between about 0.01% w/v to about 5% w/v, between about 0.05% w/v to about 0.1% w/v, between about 0.05% w/v to about 0.5% w/v, between about 0.05% w/v to about 1% w/v, between about 0.05% w/v to about 2% w/v, between about 0.05% w/v to about 3% w/v, between about 0.05% w/v to about 4% w/v, between about 0.05% w/v to about 5% w/v, between about 0.1% w/v to about 0.5% w/v, between about 0.1% w/v to about 1% w/v, between about 0.1% w/v to about 2% w/v, between about 0.1% w/v to about 3% w/v, between about 0.1% w/v to about 4% w/v, between about 0.1% w/v to about 5% w/v, between about 0.5% w/v to about 1% w/v, between about 0.5% w/v to about 2% w/v, between about 0.5% w/v to about 3% w/v, between about 0.5% w/v to about 4% w/v, between about 0.5% w/v to about 5% w/v, between about 1% w/v to about 2% w/v, between about 1% w/v to about 3% w/v, between about 1% w/v to about 4% w/v, between about 1% w/v to about 5% w/v, between about 2% w/v to about 3% w/v, between about 2% w/v to about 4% w/v, between about 2% w/v to about 5% w/v, between about 3% w/v to about 4% w/v, between about 3% w/v to about 5% w/v, or about 4% w/v to about 5% w/v, between about 0.01% w/v, between about 0.05% w/v, between about 0.1% w/v, between about 0.5% w/v, between about 1% w/v, between about 2% w/v, between about 3% w/v, between about 4% w/v, or about 5% w/v. In some embodiments, the anti-acne medication is selected from tetracycline, retinoids, salicylic acid and benzoyl peroxide.

In certain embodiments, the compositions and formulations described herein comprise one or more anti-acne medication at a concentration of about 0.01% w/v, about 0.05% w/v, about 0.1% w/v, about 0.5% w/v, about 1% w/v, about 2% w/v, about 3% w/v, or about 4% w/v, about 5% w/v, about 10% w/v, about 15% w/v, about 20% w/v, about 30% w/v, about 40% w/v, about 50% w/v, about 60% w/v, about 70% w/v, about 80% w/v, about 90% w/v, or about 99% w/v. In some embodiments, the anti-acne medication is selected from tetracycline, retinoids, salicylic acid and benzoyl peroxide.

In some embodiments, compositions and formulations described herein comprise one or more anti-acne medications. In some embodiments, the concentration of the one or more anti-acne medications is determined by the machine learning algorithm. In some embodiments, compositions and formulations described herein comprise two or more anti-acne medications. In some embodiments, the concentration of each anti-acne medication is determined by the machine learning algorithm.

In some embodiments, the compositions and formulations described herein are in the form of a lotion, emulsion, cream, ointment, gel, foam, or emollient.

A lotion may be described as a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

An emulsion may be described as a preparation of one liquid distributed in small globules throughout the body of a second liquid. In some embodiments, the dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as polyethylene glycol. The oil phase may contain other oily pharmaceutically approved excipients. For example, materials such as hydroxylated castor oil or sesame oil may be used in the oil phase as surfactants or emulsifiers.

A cream may be described as a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An ointment may be described as a semisolid preparation containing an ointment base and optionally one or more active agents of this disclosure. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A gel may be described as a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alkene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams may be described as an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Emollients may be described as externally applied agents that soften or soothe skin and are generally known in the art and listed in compendia, such as the “Handbook of Pharmaceutical Excipients”, 4.sup.th Ed., Pharmaceutical Press, 2003. In certain embodiments, the emollients are almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

Surfactants are surface-active agents that lower surface tension and thereby increase the emulsifying, foaming, dispersing, spreading and wetting properties of a product. In certain embodiments, suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

Emulsifiers are surface active substances which promote the suspension of one liquid in another and promote the formation of a stable mixture, or emulsion, of oil and water. In certain embodiments, the emulsifiers are metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof.

In some embodiments, compositions disclosed herein comprise a buffer, wherein the buffer controls a pH of the composition. Preferably, the buffers buffer the composition from a pH of about 4 to a pH of about 7.5, from a pH of about 4 to a pH of about 7, and from a pH of about 5 to a pH of about 7. In some embodiments, the buffer comprises phosphate buffered saline. In some embodiments, the buffer is phosphate buffered saline. In some embodiments, the buffer comprises acetate buffered solution. In some embodiments, the buffer is acetate buffered solution. In some embodiments, the buffer is sodium acetate buffered solution.

In some embodiments, compositions disclosed herein are formulated to provide or maintain a desirable skin pH. In some embodiments, the desirable skin pH is between about 4.5 and about 6.5. In some embodiments, the desirable skin pH is between about 5 and about 6. In some embodiments, the desirable skin pH is about 5.5. In some embodiments, compositions disclosed herein are formulated for use with a skin pH modulating agent. In some embodiments, compositions disclosed herein are formulated for use before use of a skin pH modulating agent. In some embodiments, compositions disclosed herein are formulated for use after use of a skin pH modulating agent. Non-limiting examples of pH modulating agents include salicylic acid, glycolic acid, trichloroacetic acid, azeilic acid, lactic acid, aspartic acid, hydrochloride, stearic acid, glyceryl stearate, cetyl palmitate, urea phosphate, and tocopheryl acetate. In some instances, compositions disclosed herein are formulated to lower pH of the skin of the subject to the desirable skin pH. In other words, compositions disclosed herein may comprise a pH lowering or pH balancing agent. Non-limiting examples of pH lowering and pH balancing agents include citric acid, lactic acid, glycolic acid, azelaic acid, retinoic acid, alpha hydroxyl acid, and ascorbic acid.

In some embodiments, compositions disclosed herein are formulated to provide more oxygen to the skin. In some embodiments, compositions disclosed herein are formulated to provide more oxygen exposure to the skin. In some embodiments, compositions disclosed herein are formulated to provide more oxygen diffusion into the skin. In some embodiments, compositions disclosed herein are formulated to provide more oxygen diffusion through the skin. In some embodiments, compositions disclosed herein are formulated with an agent that provides more oxygen to the skin. In some embodiments, compositions disclosed herein are used with an agent that provides more oxygen to the skin. In some embodiments, compositions disclosed herein are used before use of an agent that provides more oxygen to the skin. In some embodiments, compositions disclosed herein are used after use of an agent that provides more oxygen to the skin. A non-limiting example of an agent that provides oxygen to the skin is chlorophyll.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal. In one embodiment, a concentration of a preservative that is effective to prevent fungal growth is selected, without affecting the effectiveness of the composition for its intended purposed upon topical application.

Excipients in the formulation are selected based on the type of formulation intended. In certain embodiments, the excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars, and starches.

After formulation, composition disclosed herein may be packaged in a manner suitable for delivery and use by an end user. In one embodiment, the composition is placed into an appropriate dispenser and shipped to the end user. Examples of a final container may include a pump bottle, squeeze bottle, jar, tube, capsule or vial.

In some embodiments, compositions disclosed herein can be added to an applicator before packaging. Non-limiting examples of applicators include a cotton pad, a polyester pad, a q-tip, a sponge, and a brush. In some instances, the applicator is a cotton pad. Advantageously, a cotton pad does not bind bacteria, delivers a standardized dose, costs little, and is familiar to both clinicians and patients.

In some embodiments, pharmaceutical compositions and applicators disclosed herein are placed in a package. Non-limiting examples of a package includes bags, foil, and wax lined paper packets. In some instances, the packaging comprises plastic. In some instances, the packaging comprises a plastic bag. In some instances, the plastic comprises a material selected from polypropylene, nylon, and a combination thereof. In some instances, the plastic is thin and flexible so that it can cool, freeze, thaw or warm quickly. By way of non-limiting example, the plastic may be about 1 mil thick to about 3 mil thick (1 mil= 1/1000 of an inch). The interior of the package may be sterile. In some embodiments, air in the package is removed with a vacuum before sealing. Vacuum sealing may advantageously eliminate oxygen, increase speed of freezing (increases viability after frozen storage), ensure uniform distribution of formulation, and enable convenient compact single use container. In some embodiments, the package is heat-sealed. In some embodiments, the package is sealed with adhesive.

In another embodiment, compositions disclosed herein are lyophilized, spray-dried or freeze dried, for reconstitution before application to the skin. By way of non-limiting example, reconstitution may comprise wetting the composition before application to the skin. In some instances, the lyophilized, spray-dried or freeze dried composition is on an applicator, and the composition together with the applicator are reconstituted by wetting the applicator. In some instances, wetting occurs by applying water to the skin and then applying the applicator and/or composition to the wet skin. In some embodiments, the composition does not have to be frozen.

Compositions disclosed herein may comprise additional active ingredients. In certain embodiments, compositions disclosed herein comprise a constituent may be selected from a small molecule, a fatty acid, an antibiotic, a metabolite, an antioxidant, and a retinoid. Non-limiting examples of antibiotics include benzoyl peroxide, salicylic acid, tetracycline, macrolide, β-lactam, aminoglycoside, cephalosporin, carbapenems, quinolone/fluoroquinolone, sulfonamides, salicylic acid, glycolic acid, azaleic acid, live phage therapy, synthetic phage contractile nanotubes, laser, dapsone, benzoyl peroxide, benzoyl peroxide/resveratrol combinations, and any combination thereof. In some embodiments, the antibiotic is selected from clindamycin, doxycycline, erythromycin, and tetracycline, wherein the antibiotic is formulated for topical administration. Non-limiting examples of antioxidants are vitamin C and vitamin E. Non-limiting examples of a retinoid are tretinoin, retinoic acid, tazarotene, adapalene, and retinol.

In certain embodiments, a composition disclosed herein comprises anti-acne medication. In some embodiments, the concentration of the anti-acne medication is between about 0.1% and about 3%. In some embodiments, the concentration of the anti-acne medication is between about 0.1% and about 2.5%. In some embodiments, the concentration of the anti-acne medication is between about 0.5% and about 2.5%. In some embodiments, the concentration of the anti-acne medication is between about 1% and about 2.5%. In some embodiments, the concentration of the anti-acne medication is less than about 2.5%. In some embodiments, the concentration of the anti-acne medication is less than about 2%. In some embodiments, the concentration of the anti-acne medication is less than about 1.5%. In some embodiments, the concentration of the anti-acne medication is less than about 1%. In some embodiments, the concentration of the anti-acne medication is less than about 0.5%. In some embodiments, the concentration of the anti-acne medication is less than about 0.1%.

In certain embodiments, a composition disclosed herein comprises benzoyl peroxide. In some embodiments, the concentration of the benzoyl peroxide is between about 0.1% and about 3%. In some embodiments, the concentration of the benzoyl peroxide is between about 0.1% and about 2.5%. In some embodiments, the concentration of the benzoyl peroxide is between about 0.5% and about 2.5%. In some embodiments, the concentration of the benzoyl peroxide is between about 1% and about 2.5%. In some embodiments, the concentration of the benzoyl peroxide is less than about 2.5%. In some embodiments, the concentration of the benzoyl peroxide is less than about 2%. In some embodiments, the concentration of the benzoyl peroxide is less than about 1.5%. In some embodiments, the concentration of the benzoyl peroxide is less than about 1%. In some embodiments, the concentration of the benzoyl peroxide is less than about 0.5%. In some embodiments, the concentration of the benzoyl peroxide is less than about 0.1%.

In certain embodiments, a composition disclosed herein comprises salicylic acid. In some embodiments, the concentration of the salicylic acid is between about 0.1% and about 3%. In some embodiments, the concentration of the salicylic acid is between about 0.1% and about 2.5%. In some embodiments, the concentration of the salicylic acid is between about 0.5% and about 2.5%. In some embodiments, the concentration of the salicylic acid is between about 1% and about 2.5%. In some embodiments, the concentration of the salicylic acid is less than about 2.5%. In some embodiments, the concentration of the salicylic acid is less than about 2%. In some embodiments, the concentration of the salicylic acid is less than about 1.5%. In some embodiments, the concentration of the salicylic acid is less than about 1%. In some embodiments, the concentration of the salicylic acid is less than about 0.5%. In some embodiments, the concentration of the salicylic acid is less than about 0.1%.

In certain embodiments, a composition disclosed herein comprises tetracycline. In some embodiments, the concentration of the tetracycline is between about 0.1% and about 3%. In some embodiments, the concentration of the tetracycline is between about 0.1% and about 2.5%. In some embodiments, the concentration of the tetracycline is between about 0.5% and about 2.5%. In some embodiments, the concentration of the tetracycline is between about 1% and about 2.5%. In some embodiments, the concentration of the tetracycline is less than about 2.5%. In some embodiments, the concentration of the tetracycline is less than about 2%. In some embodiments, the concentration of the tetracycline is less than about 1.5%. In some embodiments, the concentration of the tetracycline is less than about 1%. In some embodiments, the concentration of the tetracycline is less than about 0.5%. In some embodiments, the concentration of the tetracycline is less than about 0.1%.

In certain embodiments, compositions disclosed herein comprise a topical anti-acne medication such as a retinoid. Non-limiting examples of topical retinoid compounds include retinoic acid, tretinoin, adapalene, retinol, and tazarotene. In certain embodiments, compositions disclosed herein comprise resveratrol or trans-resveratrol. In some embodiments, the concentration of the retinoid or resveratrol in the composition is less than about 10%. In some embodiments, the concentration of the retinoid or resveratrol in the composition is less than about 5%. In some embodiments, the concentration of the retinoid or resveratrol in the composition is less than about 2.5%. In some embodiments, the concentration of the retinoid or resveratrol in the composition is less than about 1%. In some embodiments, the concentration of the retinoid or resveratrol in the composition is less than about 0.5%. In some embodiments, the concentration of the retinoid or resveratrol in the composition is between about 0.5% and about 10%. In some embodiments, the concentration of the retinoid or resveratrol in the composition is between about 1% and about 10%. In some embodiments, the concentration of the retinoid or resveratrol in the composition is between about 0.5% and about 2.5%.

In some embodiments, compositions disclosed herein comprise at least one omega-3 fatty acid. Non-limiting examples of omega-3 fatty acids include hexadecatrienoic acid (HTA), α-Linolenic acid (ALA), stearidonic acid (SDA), eicosatrienoic acid (ETE), eicosatetraenoic acid (ETA), eicosapentaenoic acid (EPA), heneicosapentaenoic acid (HPA), docosapentaenoic acid (DPA), clupanodonic acid, docosahexaenoic acid (DHA), tetracosapentaenoic acid, tetracosahexaenoic acid (nisinic acid), and phytosphingosine.

In some embodiments, compositions disclosed herein comprise an acid selected from glycolic acid, azaelic acid, and trichloroacetic acid. In some embodiments, compositions disclosed herein comprise a natural extract, such as tea tree oil or green tea extract.

In some embodiments, the additional active ingredient comprises a drug targeting at least one strain of P. acnes. In some embodiments, the drug targeting at least one strain of P. acnes is a small molecule drug. In some embodiments, the drug targeting at least one strain of P. acnes is a small molecule inhibitor of an enzyme expressed by P. acnes. In some embodiments, the enzyme expressed by P. acnes is required for P. acnes growth or P. acnes energy metabolism.

### EXAMPLES

The following examples and figures are provided to further illustrate the embodiments of the present invention, but are not intended to limit the scope of the invention. While they are typical of those that might be used, other procedures, methodologies, or techniques known to those skilled in the art may alternatively be used.

Described herein are systems, methods, and compositions for treating a skin condition in an individual. A system or method is used to determine a skin flora of an individual and a sensitivity of the skin flora to a treatment modality. A composition is then customized based on the sensitivity of the skin flora to the treatment modality.

Described herein are findings of a study of individuals comprising preadolescent females with acne, and changes in the microbiomes of the individuals with the use of benzoyl peroxide wash. Flora was taken from different areas of the faces of the individuals and studied for sensitivity to benzoyl peroxide.

### Example 1—Benzoyl Peroxide Sensitivity

Inclusion criteria of the study for which results are provided herein include:
1. Minimum of 6 acne lesions present.
2. No prior use of prescription acne product.
3. No prior use of over-the-counter benzoyl peroxide anti-acne agent.
4. Ages: 7-12
5. Sex: Female

Exclusion criteria of the study for which results are provided herein include:
1. Significant active skin conditions on the face other than acne vulgaris (may include atopic dermatitis, psoriasis, or others).
2. Use of oral or parenteral antibiotics within 3 months prior to the study.

Sites from which samples were taken along with abbreviations for each include:
RA or RAL: Retroauricular Behind Ear
CH or CHN: Chin
CK or CKN: Cheek
FH or FHN: Forehead
AN or ANN: Ala Nasal [Outside of Nose]
CO or CON: Comedone

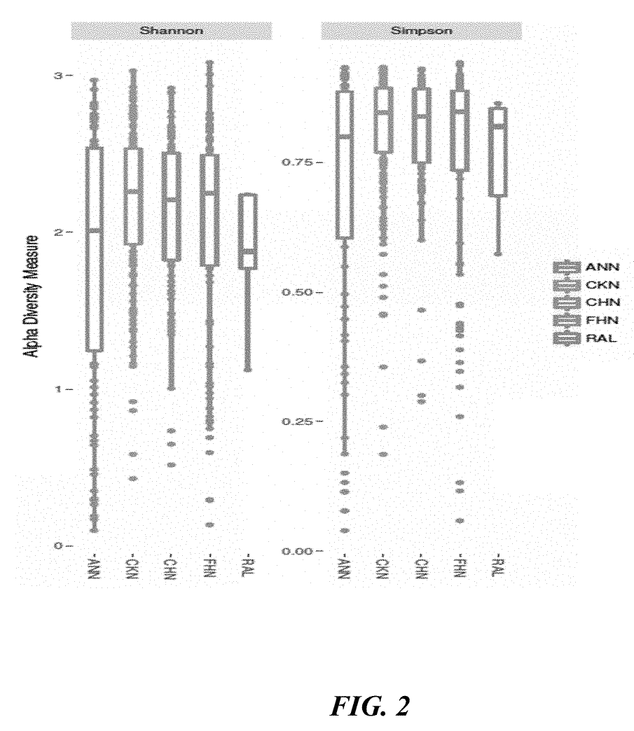

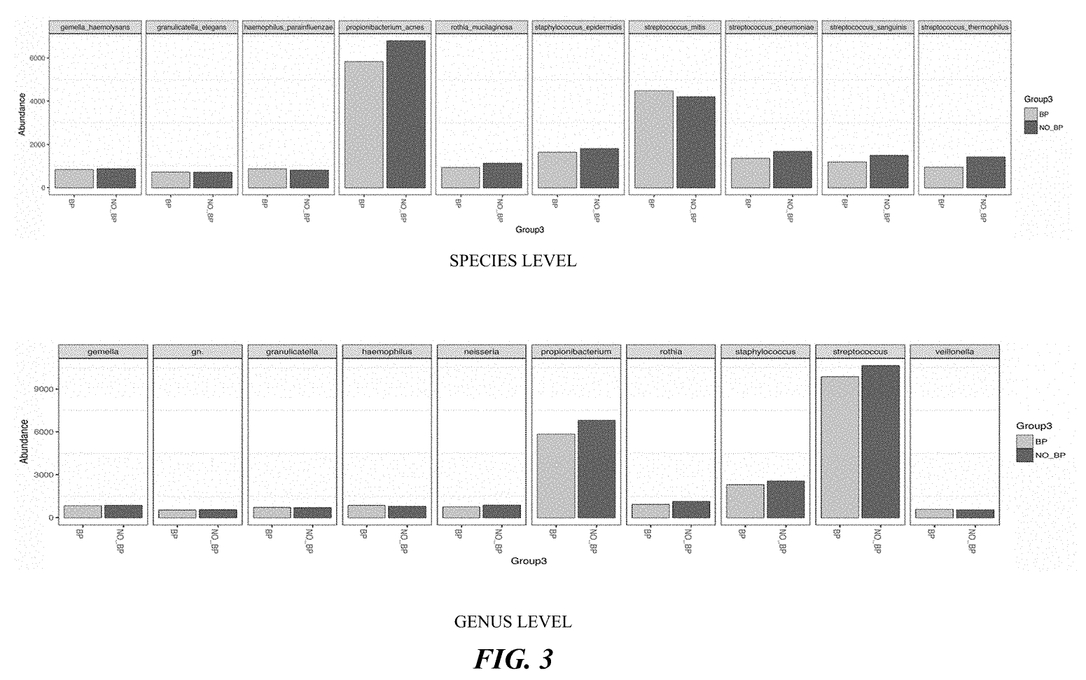

In some embodiments, the skin microbiome of an individual subject is evaluated. Individual microbiomes vary greatly across subjects and regions of the skin. FIG. 2 shows flora diversity at different locations on the skin surfaces of two different individuals Shannon and Simpson. Samples were taken from locations on the heads of the individuals including behind the ear, chin, cheek, forehead, and outside the nose. As shown, the diversity at the various locations differed between individuals Shannon and Simpson. With application of an anti-microbial agent such as benzoyl peroxide, flora that is more sensitive to the treatment decrease within the microflora. This has an overall effect on the microbiome, wherein species sensitive to the treatment modality used decrease within the microbiome of the individual while those flora with less sensitivity to the treatment tend to increase within the microbiome. FIG. 3 shows multiple graphs of subsets of microbiomes in which benzoyl peroxide was applied and those where no benzoyl peroxide was applied and shows the relative proportion of the flora in each group of benzoyl peroxide and no benzoyl peroxide. As shown, with benzoyl peroxide application, P. acnes levels within the microbiome decreased. It is also notable that with benzoyl peroxide, S. mitis increased. Table 2 shows findings for site specific (i.e. specific region of skin) data of benzoyl peroxide versus no benzoyl peroxide. To assess site specific effects to benzoyl peroxide versus no benzoyl peroxide a prediction model was created for each site for each site that can evaluate whether the site has been exposed to benzoyl peroxide or not. As shown in Table 2, the best performance (i.e. prediction) is observed for the region of the cheek which indicates that cheek gets impacted the most by benzoyl peroxide.

TABLE 2

| Site | P-value | Accuracy | # of Samples |
|---|---|---|---|
| Outside of Nose | 0.987 | 44% | 99 |
| Comedone | 0.762 | 35% | 61 |
| Forehead | 0.994 | 35% | 98 |
| Behind Ear | 0.054 | 65% | 89 |
| Cheek | 0.001 | 82% | 198 |
| Combined | 0.962 | 67% | 545 |

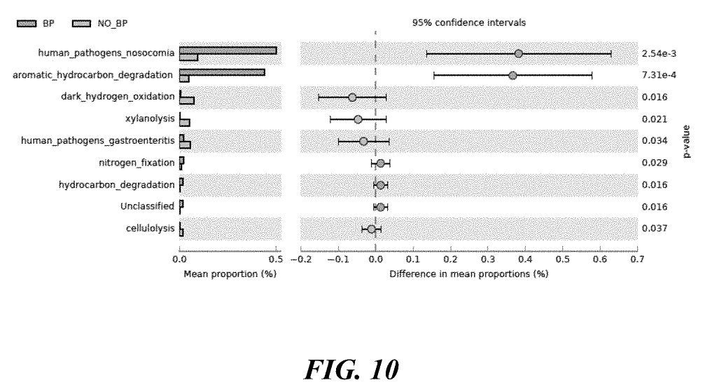

In some embodiments, analysis of a subject skin microbiome requires the evaluation of biomarkers and functions produced by the microbiome. FIG. 10 shows a comparative functional analysis for flora on skin regions to which benzoyl peroxide was applied and flora on skin regions to which no benzoyl peroxide was applied. FIG. 10 shows the effect of benzoyl peroxide on the production of biomarkers by the skin microbiome.

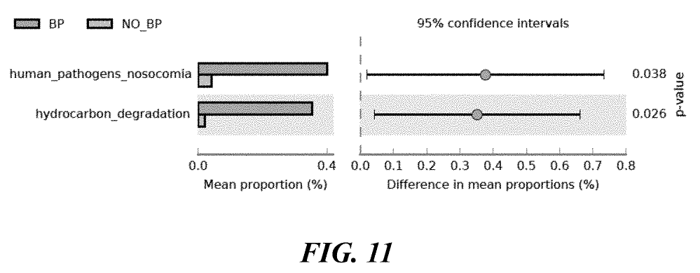

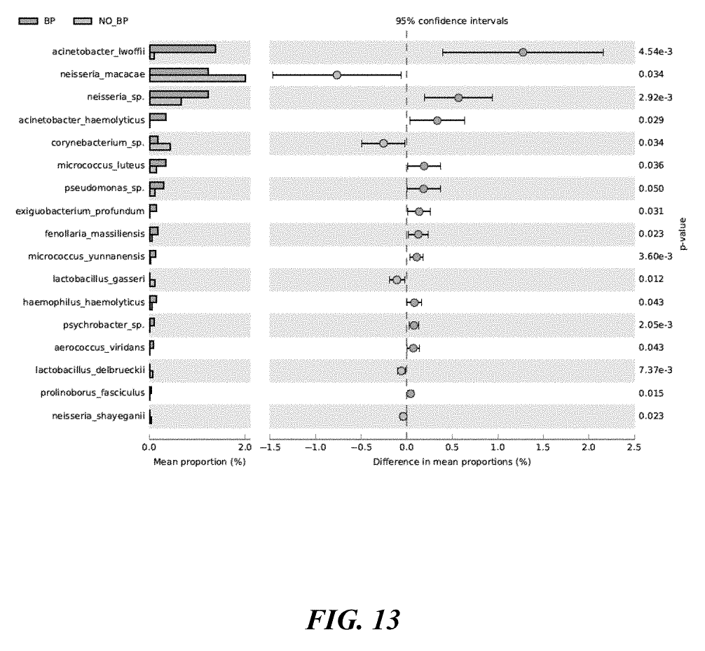

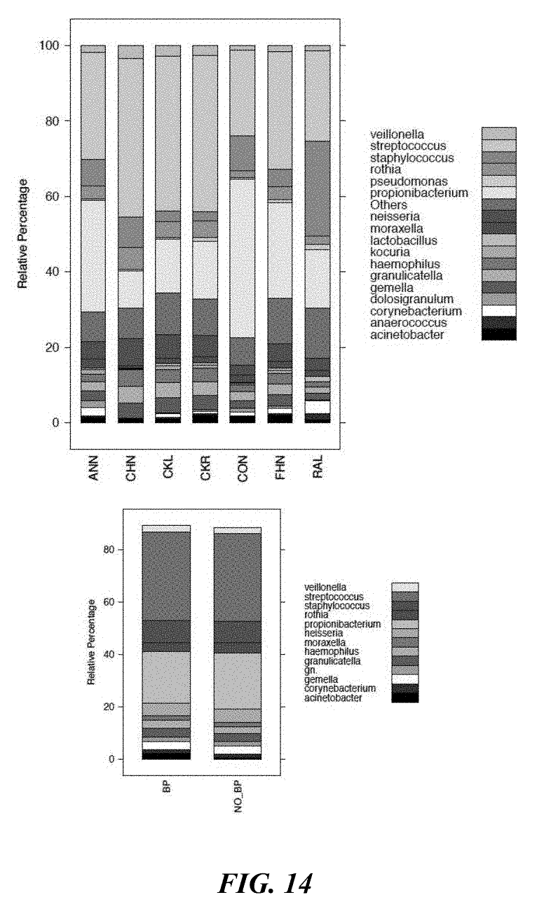

In some embodiments, analysis of a subject skin microbiome requires the identification and enumeration of one or more microflora. FIG. 11 shows a comparative functional analysis for flora on the cheek to which benzoyl peroxide was applied and flora on the cheek to which no benzoyl peroxide was applied. Redoing the functional analysis between the two visits just on cheek samples yields the same functional categories. FIG. 13 shows a representation of total diversity at the genus level for site specific and treatment specific results (i.e. with benzoyl peroxide and with no benzoyl peroxide). FIG. 14 shows a representation of total diversity at the species level for site specific and treatment specific results (i.e. with benzoyl peroxide and with no benzoyl peroxide), as well as the average of all sites.

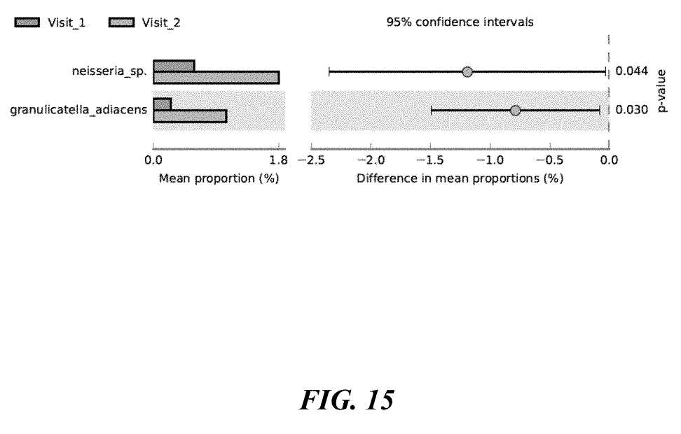

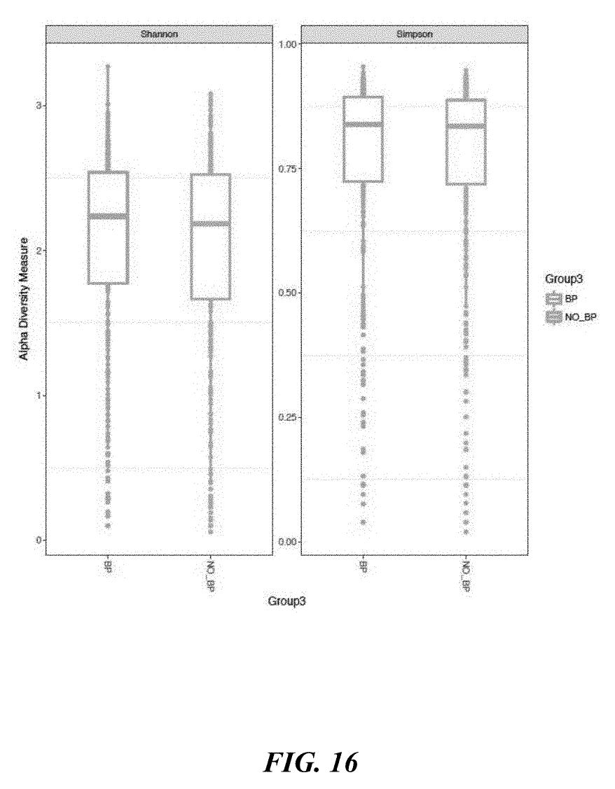

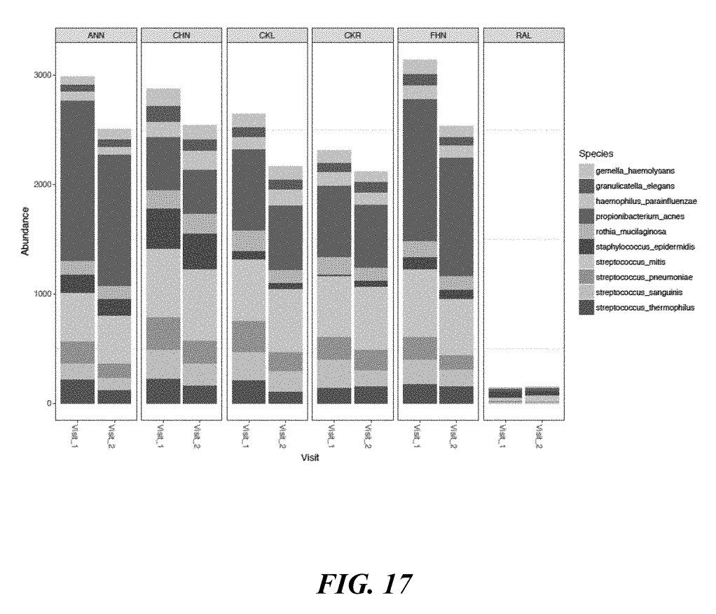

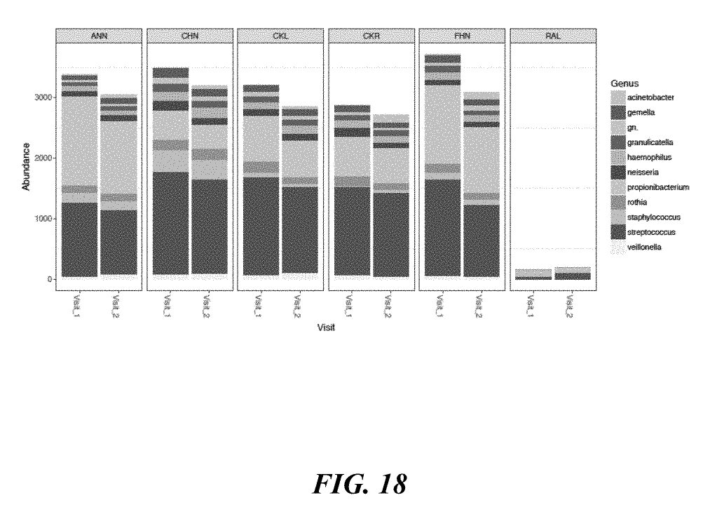

FIG. 15 shows a comparison of statistically significant taxa between two different samples taken from different times (visits) where samples were taken from the cheek. Species were shown to increase from a first visit to a second visit. Following the first visit, benzoyl peroxide was applied to the site. FIG. 16 shows diversity measures both before and after benzoyl peroxide is added to a skin region. FIG. 17 shows taxonomic genera with a significant change between two different visits at a species level. Taxonomic species that changed significantly between the two visits are shown, broken down based on the site. FIG. 18 shows taxonomic genera with a significant change between two different visits at a genus level. Taxonomic species that changed significantly between the two visits are shown, broken down based on the site.

### Machine Learning Algorithm

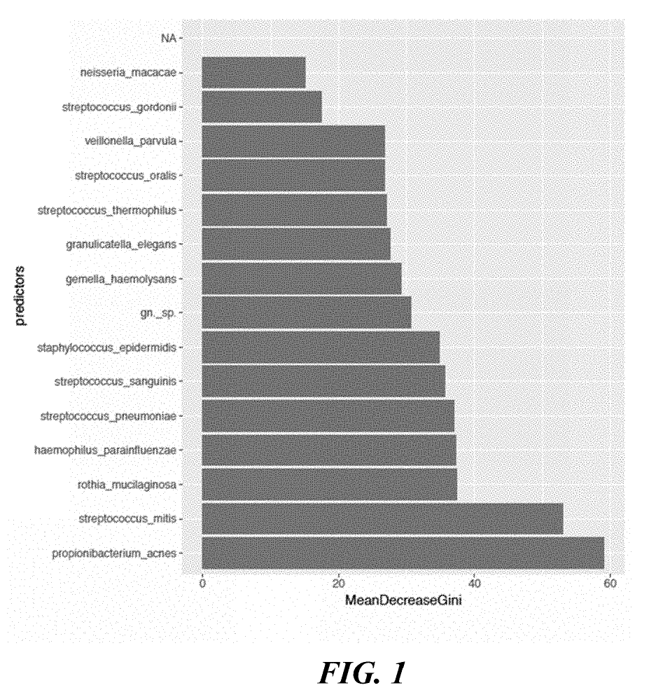

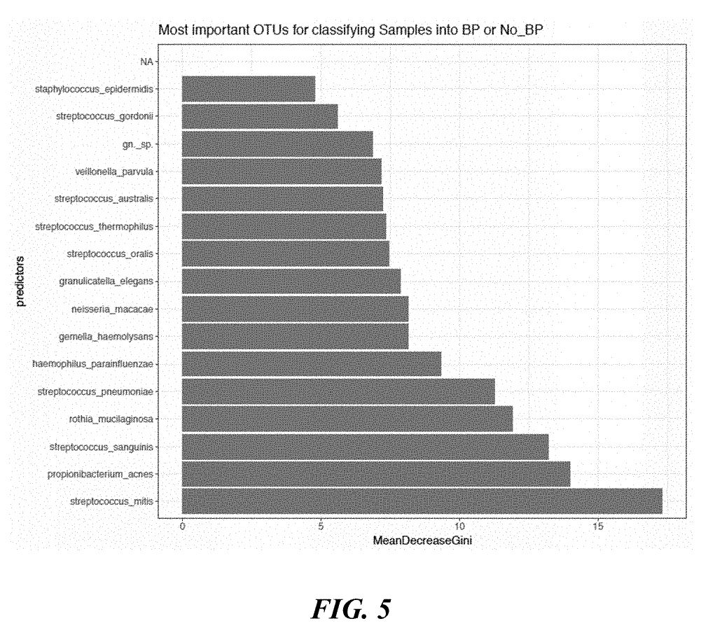

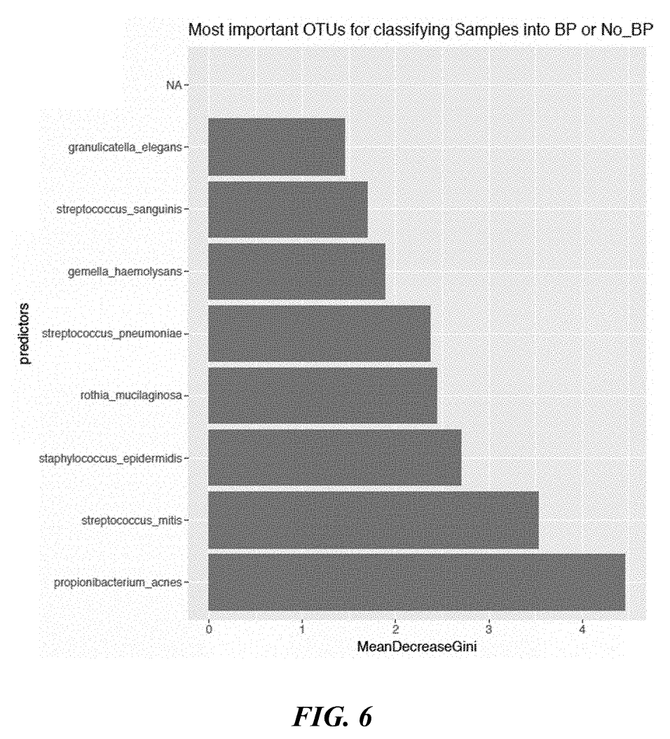

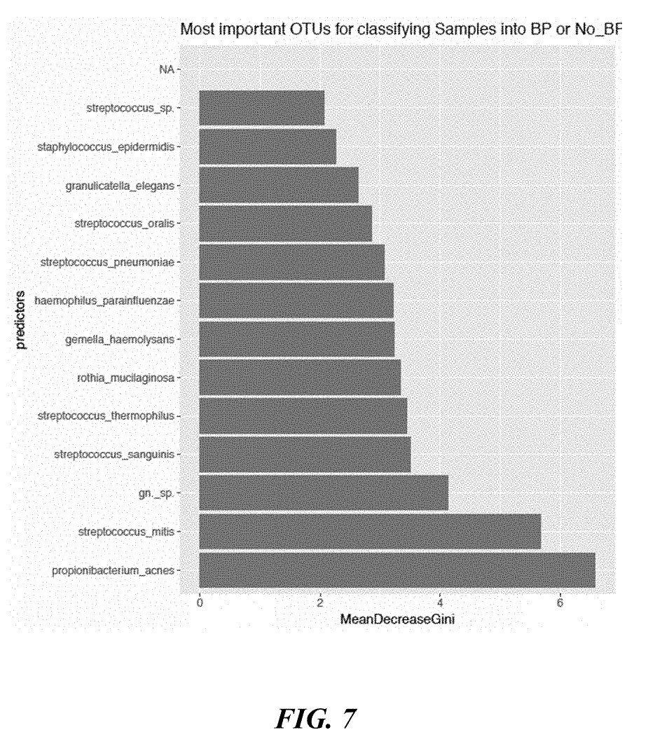

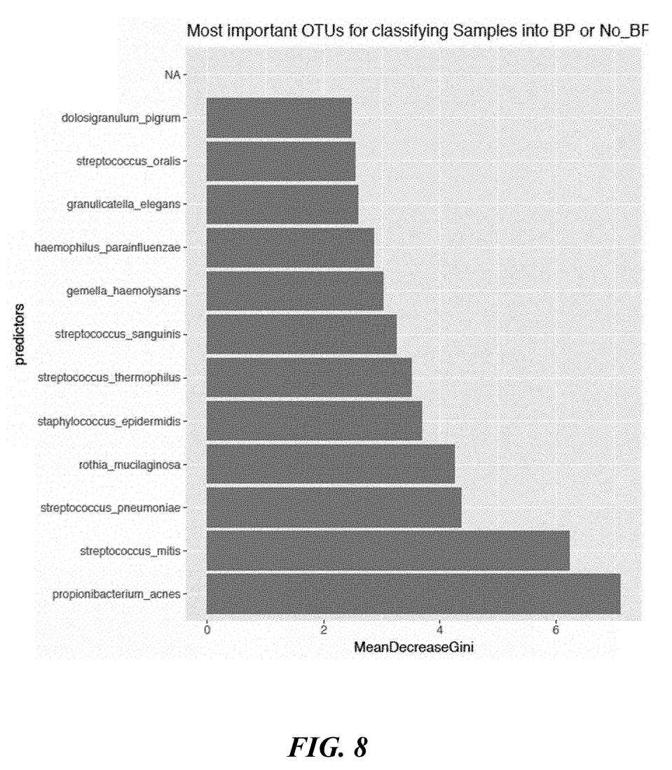

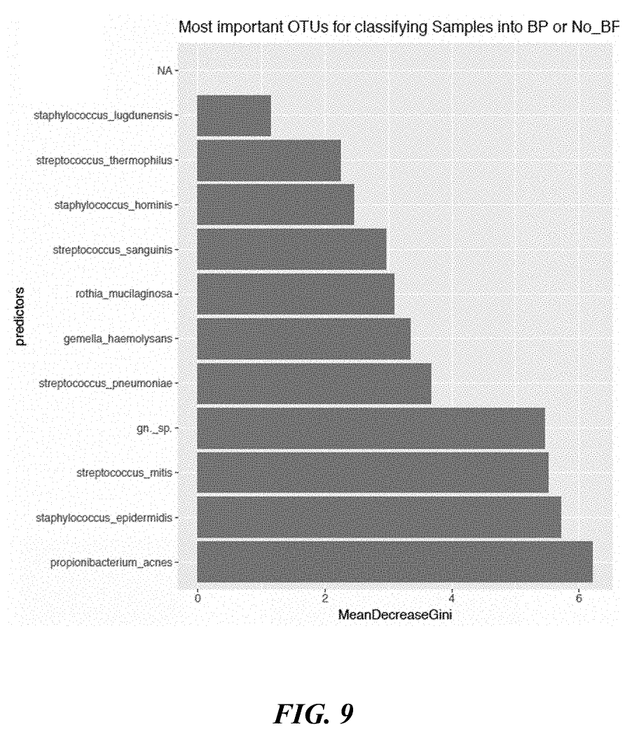

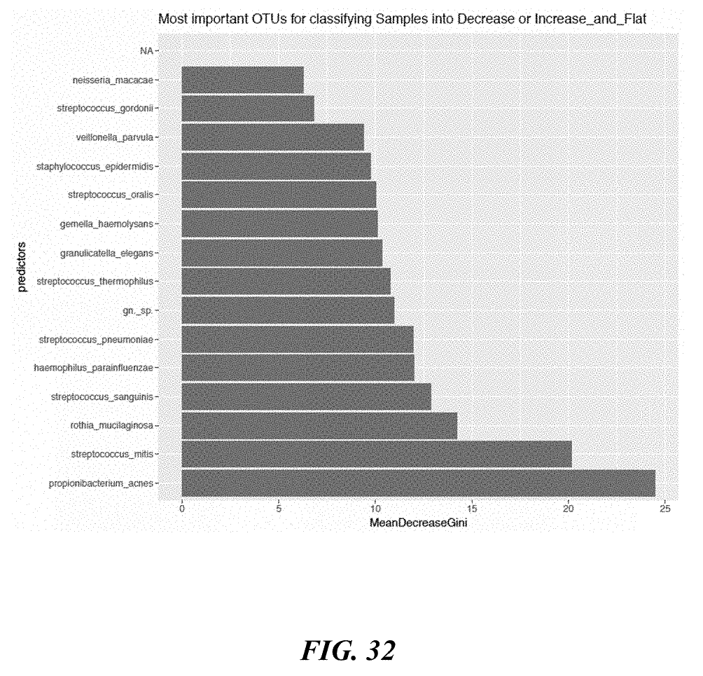

Clinical tests measuring the efficacy of benzoyl peroxide for the treatment of acnes vulgaris were utilized to build a machine learning model. The machine learning model was built to predict whether an individual will respond positively to benzoyl peroxide or not with 75% accuracy. The model can be de-constructed to identify the top predictors shown in FIGS. 1, 5-9, and 32. FIG. 1 shows a graph of the predicted flora on the skin surface of an individual affected by acne where the predicted flora are represented on the x-axis and the Mean Decrease Gini is represented on the y-axis. As shown, P. acnes is expected to have the highest Mean Decrease Gini followed by S. mitis. FIG. 5 shows a graph of species used to determine whether any skin region was exposed to benzoyl peroxide or no benzoyl peroxide. As shown, S. mitis had the highest Mean Decrease Gini value and P. acnes had the second highest. FIG. 6 shows a graph of species used to determine whether a skin region comprising a comedone was exposed to benzoyl peroxide or no benzoyl peroxide. As shown, P. acnes had the highest Mean Decrease Gini value and S. mitis had the second highest. FIG. 7 shows a graph of species used to determine whether a skin region comprising a forehead was exposed to benzoyl peroxide or no benzoyl peroxide. As shown, P. acnes had the highest Mean Decrease Gini value and S. mitis had the second highest. FIG. 8 shows a graph of species used to determine whether a skin region comprising the outside of the nose was exposed to benzoyl peroxide or no benzoyl peroxide. As shown, P. acnes had the highest Mean Decrease Gini value and S. mitis had the second highest. FIG. 9 shows a graph of species used to determine whether a skin region comprising behind the ear was exposed to benzoyl peroxide or no benzoyl peroxide. As shown, P. acnes had the highest Mean Decrease Gini value and S. mitis had the second highest. FIG. 32 shows a summary graph representing the flora that are the top predictors for providing a prediction of a response to benzoyl peroxide based on the microbiome of the individual.

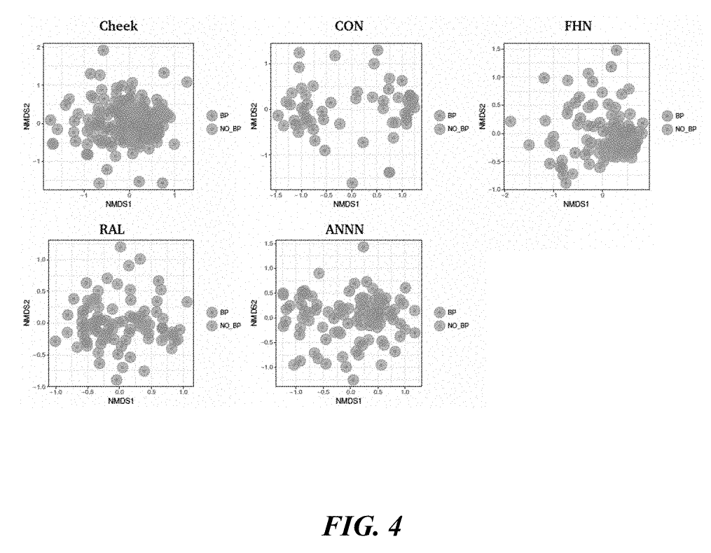

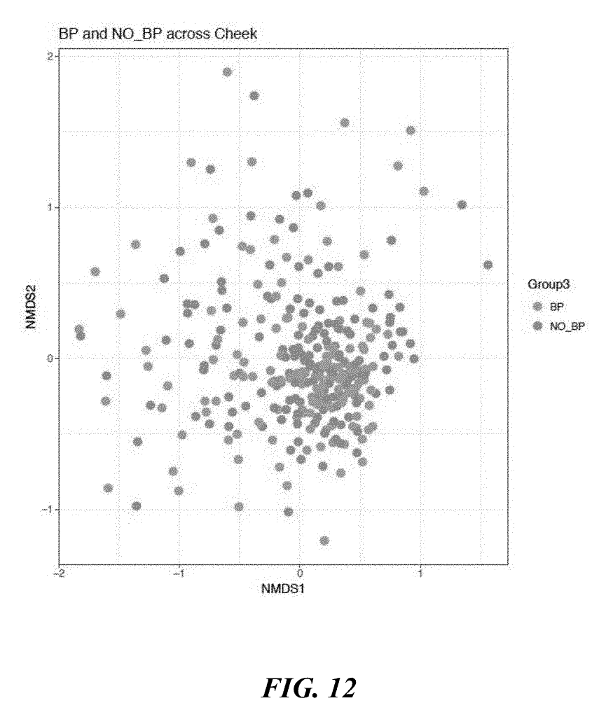

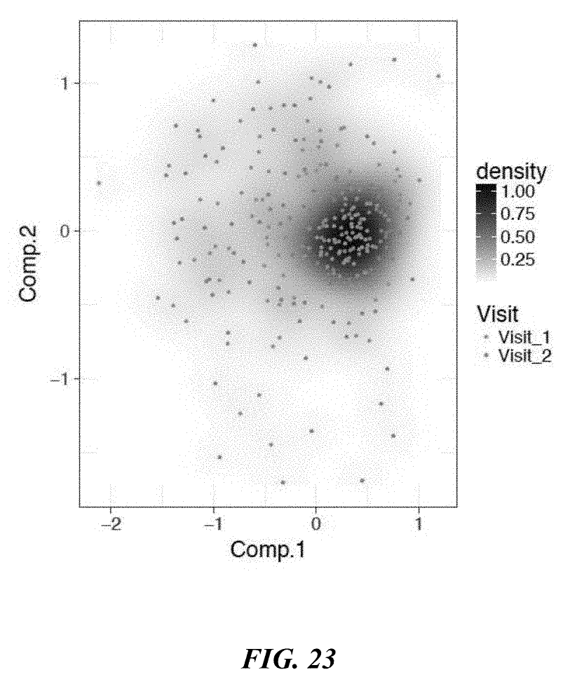

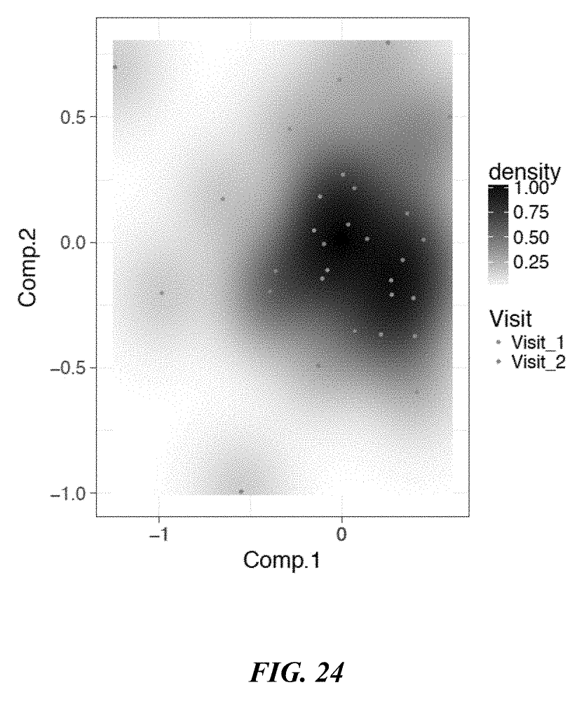

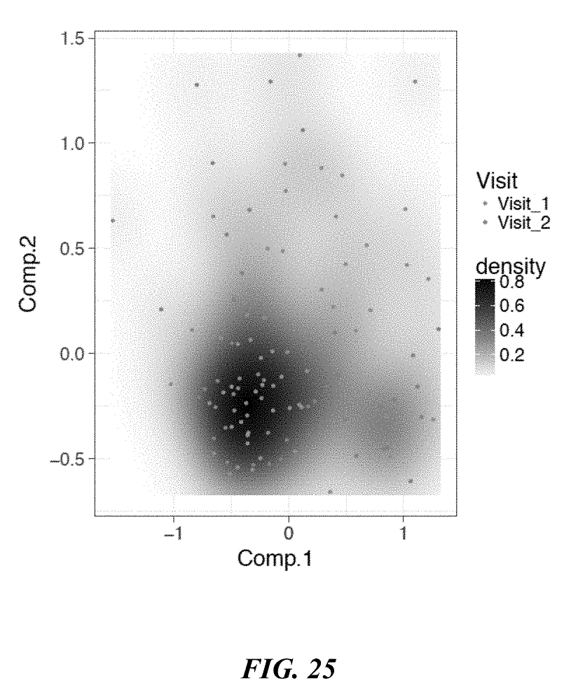

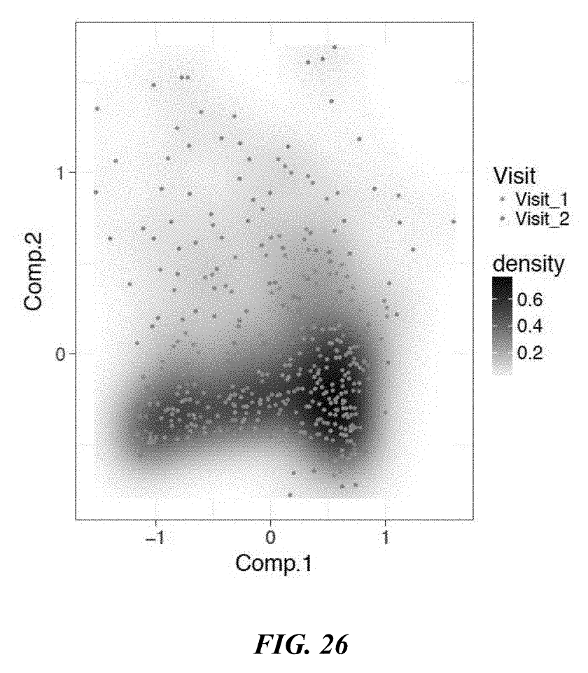

In some embodiments, metabolic modelling using machine learning algorithms is utilized to predict the efficacy of one or more medicaments for the modulation of a particular subject's skin microbiome. In some embodiments, the use of traditional analytical techniques do not allow for meaningful analysis or lack predictive power. For example, FIGS. 4, 12, 23-26 depict traditional analytical techniques that were less useful in predicting the effects of the application of benzoyl peroxide to a subject's skin microbiome. FIG. 4 shows Bray-Curtis dissimilarity plots of measurements performed specific sites, such as the cheek, forehead, outer nose, behind the ear and on a comedone of two cohorts, wherein the benzoyl peroxide was applied by the first cohort and not the second. The Bray-Curtis dissimilarity plots fail to provide meaningful predictive power. FIG. 12 is a representation of the Bray Curtis Dissimilarity for the cheek skin region. In another study, a group of subjects with acne vulgaris were treated with benzoyl peroxide and divided into 3 cohorts based on the effectiveness of the treatment. Subjects in the “Decrease Cohort” experienced a decrease in acne, subjects in the “Increase Cohort” experienced an increase in acne, and the “Flat Cohort” experienced non-measureable variance in acne. Non-metric Multi-Dimensional Scaling (NMDS) was utilized to visualize the similarity a the data set. FIG. 23 shows NMDS results for the “Decrease Cohort” across two visits. FIG. 24 shows NMDS results for a “Flat Cohort” across two visits. FIG. 25 shows NMDS results for the “Increase Cohort” across two visits. FIG. 26 shows NMDS results across all outcomes across two visits. As seen in the figures, the NMDS plots fail to provide meaningful predictive power.

### Treatment Impact

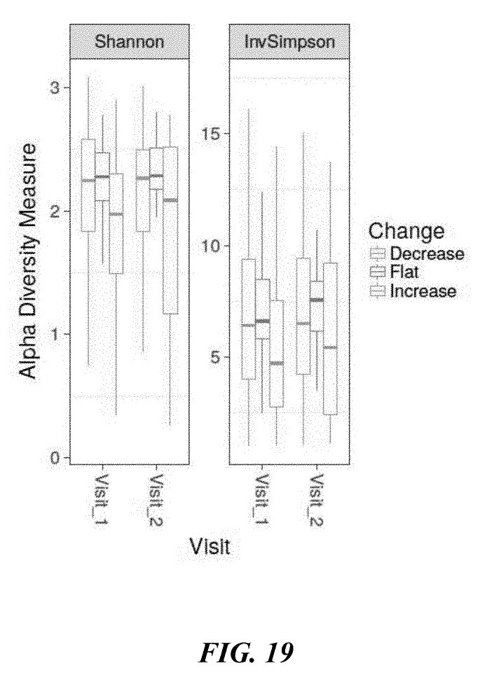

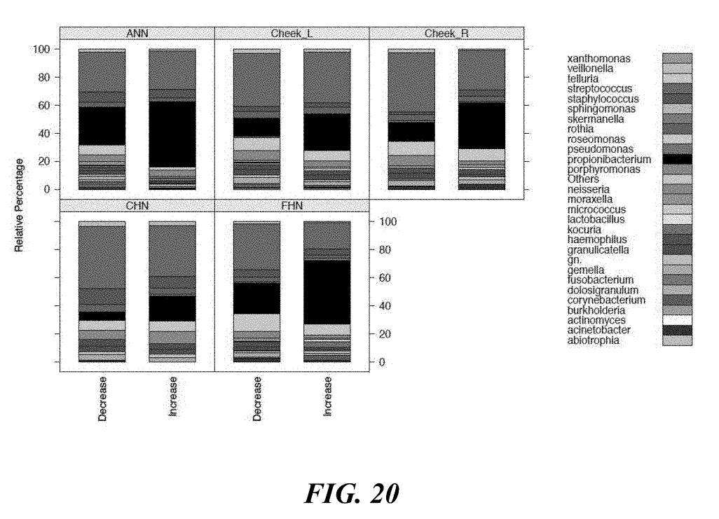

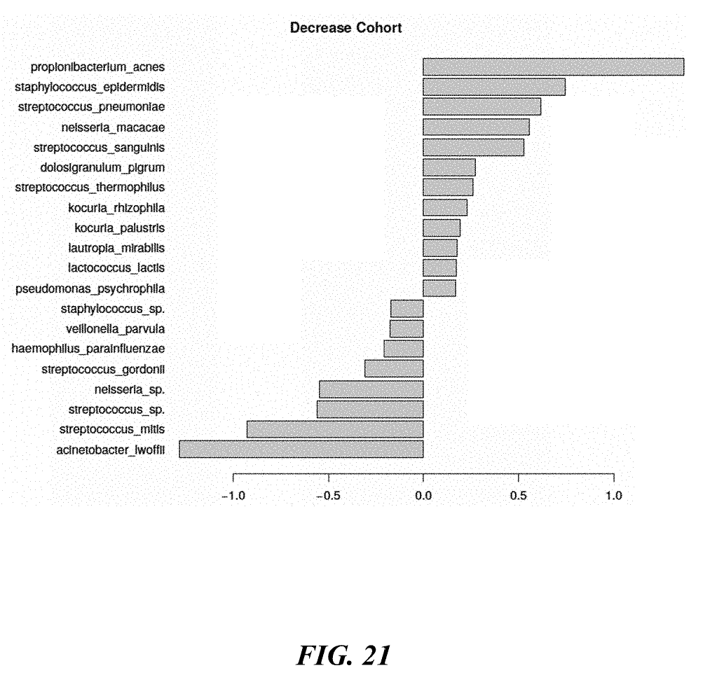

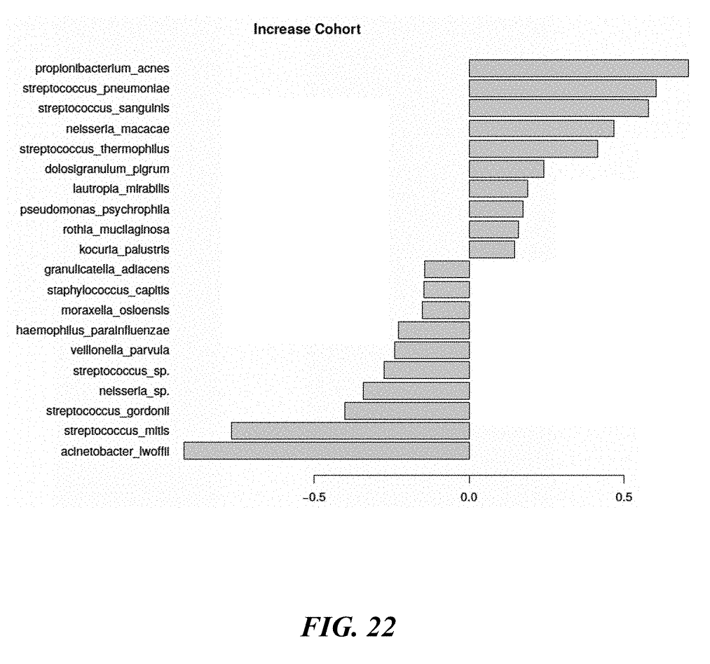

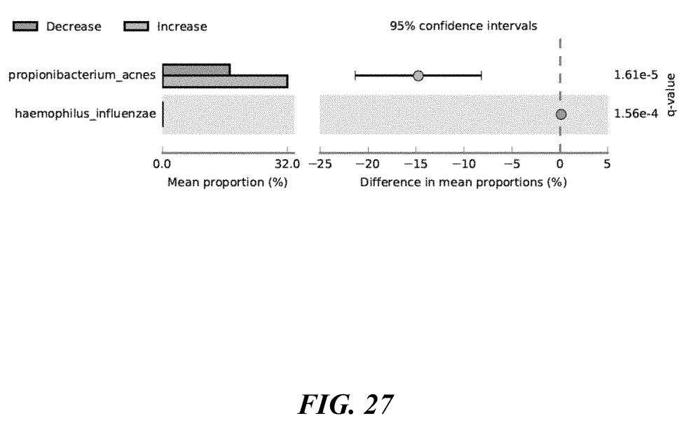

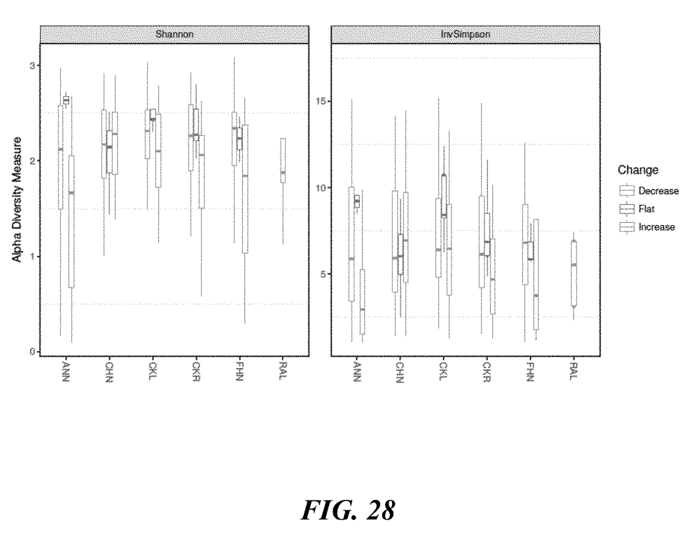

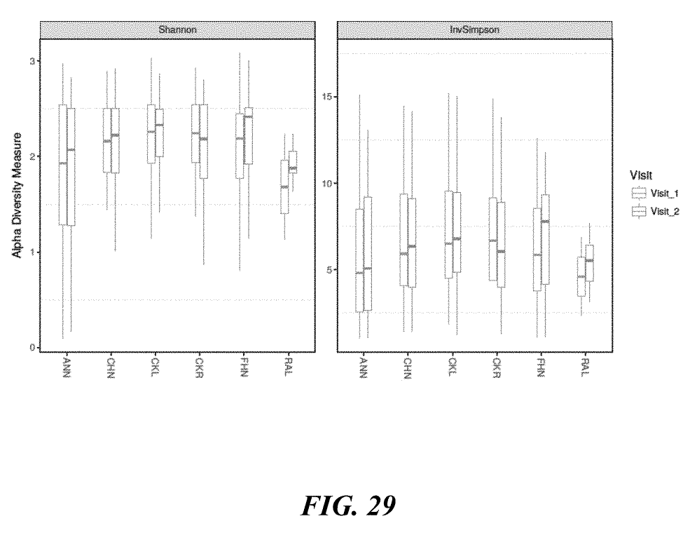

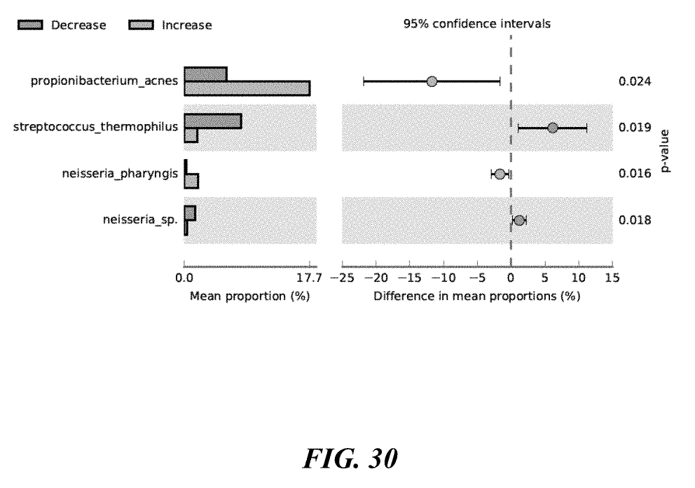

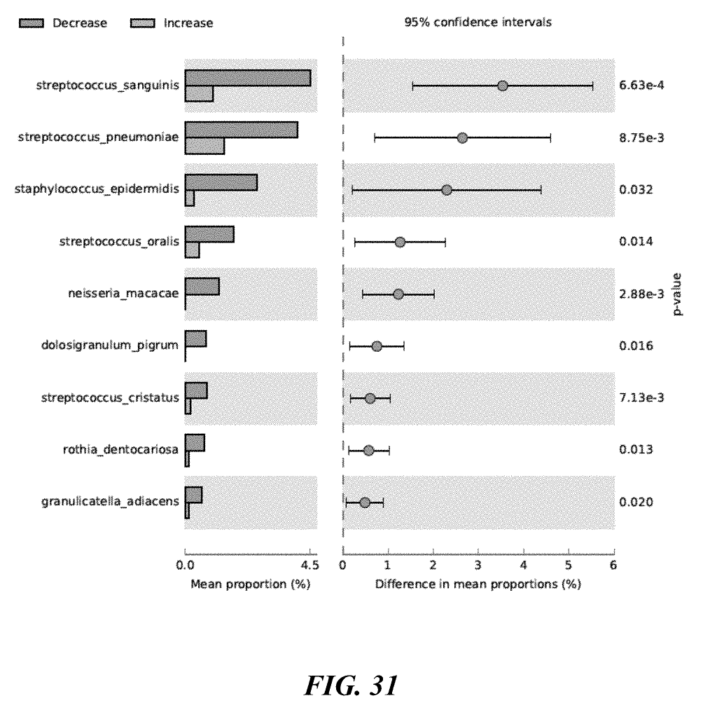

A group of subjects with acne vulgaris were treated with benzoyl peroxide and divided into 3 cohorts based on the effectiveness of the treatment. Subjects in the “Decrease Cohort” experienced a decrease in acne, subjects in the “Increase Cohort” experienced an increase in acne, and the “Flat Cohort” experienced non-measureable variance in acne. FIG. 19 shows diversity measurement results at two different visits of an individual. The average diversity increased from visit 1 to visit 2 for samples that show an increase in acne severity. FIG. 20 shows comparative microbiome profiling based on clinical outcome. P. acnes shows a significant differential representations between subjects who responded well or poorly to benzoyl peroxide. Individuals are divided based on outcome into a cohort with a response representing a decrease in acne (Decrease Cohort), and increase in acne (Increase Cohort), and essentially no response (Flat Cohort). FIG. 21 shows the top 20 taxa that can differentiate the two visits for which data is shown herein in the cohort termed the “Decrease Cohort.” FIG. 22 shows the top 20 taxa that can differentiate the two visits for which data is shown herein in the cohort termed the “Increase Cohort.” FIG. 27 shows results of a comparison between the increase and decrease cohort. The mean proportion of P. acnes higher when comparing “Increase” with “Decrease” cohort. FIG. 28 shows alpha diversity across sites based on treatment impact. FIG. 29 shows alpha diversity across sites based on visits. FIG. 30 shows the result of comparing statistically significant taxa in the Increase and Decrease Cohorts for the cheek site. Notably, S. thermophiles increased in the Decrease Cohort. FIG. 31 shows the result of comparing statistically significant taxa in the Increase and Decrease Cohorts for the forehead site. Notably, Strep and Staph species increased within the Decrease Cohort.

P. acnes

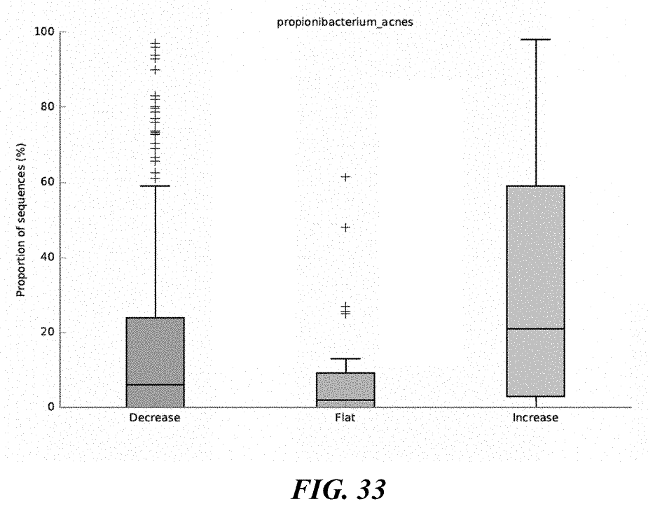

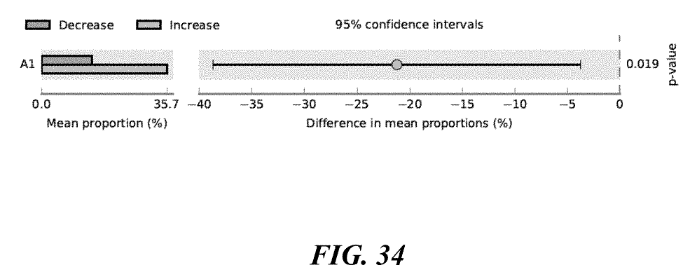

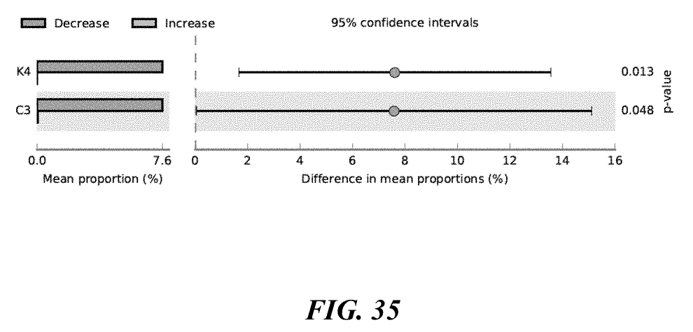

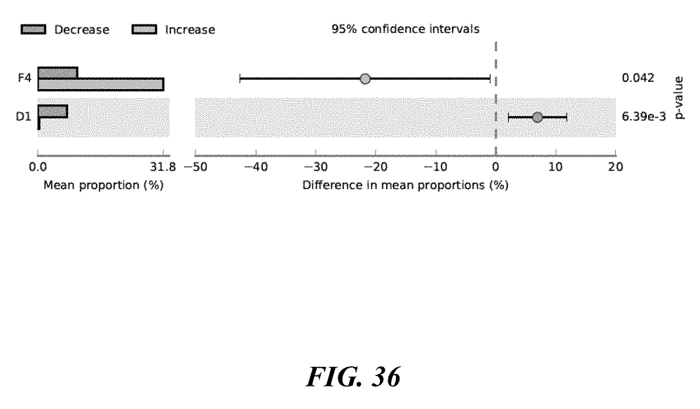

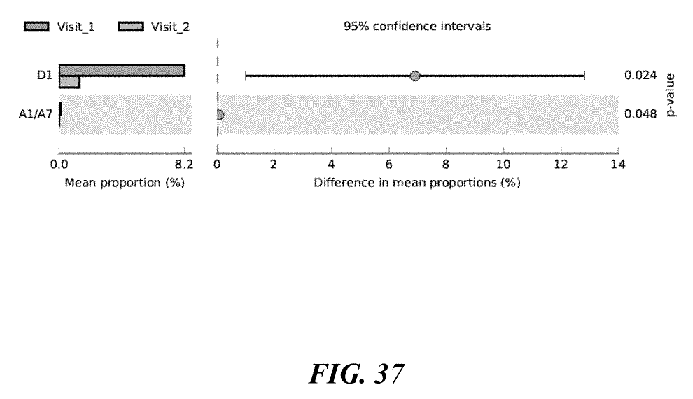

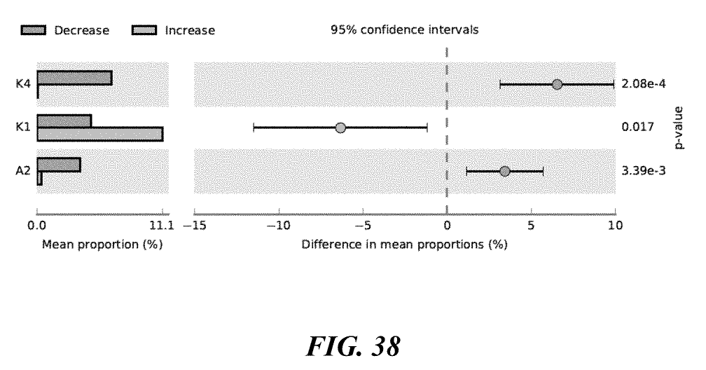

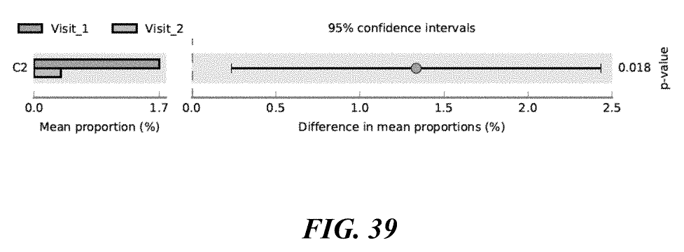

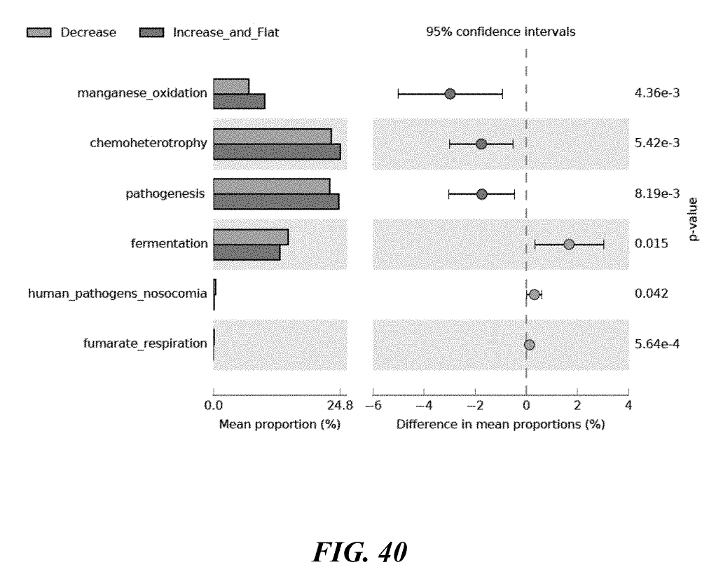

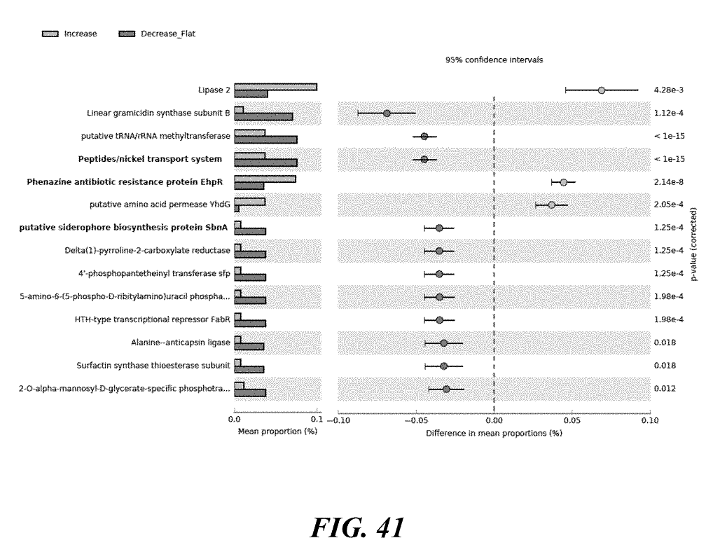

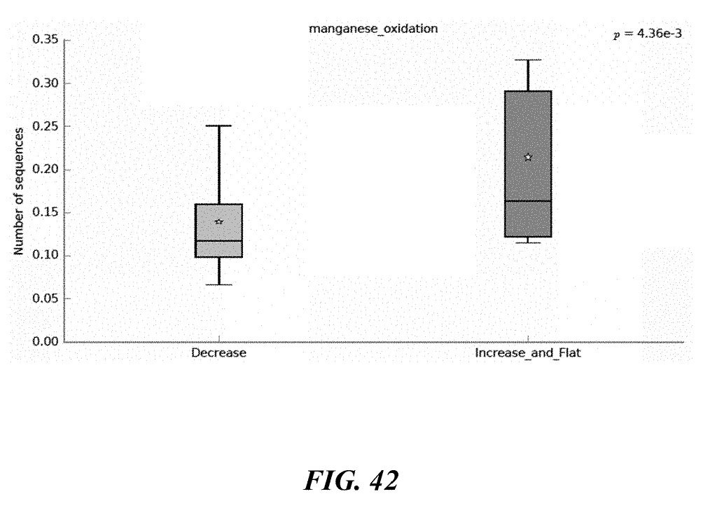

A group of subjects with P. acnes populations were treated with benzoyl peroxide and divided into 3 cohorts based on the effectiveness of the treatment. Subjects in the “Decrease Cohort” experienced a decrease in P. acnes populations, subjects in the “Increase Cohort” experienced an increase in P. acnes populations, and the “Flat Cohort” experienced non-measureable variance in P. acnes populations. FIG. 33 shows a representation of P. acnes across different groups. Shown is the mean proportion of P. acnes across “Increase”, “Decrease”, and “Flat” cohort. FIG. 34 shows P. acnes strains differentiated between Increase and Decrease Cohorts in the area outside of the nose. The Al P. acnes across is differentiated between “Increase” and “Decrease” cohort. FIG. 35 shows P. acnes strains differentiated between Increase and Decrease Cohorts in the area of the cheek. The K4 & C3 P. acnes is differentiated between “Increase” and “Decrease” cohort. FIG. 36 shows P. acnes strains differentiated between Increase and Decrease Cohorts in the area of the forehead. The F4 & D1 P. acnes is differentiated between “Increase” and “Decrease” cohort. FIG. 37 shows P. acnes strains differentiated before and after benzoyl peroxide treatment of the forehead. The F4 & D1 P. acnes is differentiated between “Increase” and “Decrease” cohort. FIG. 38 shows P. acnes strains differentiated between Increase & Decrease Cohort. The K4 and A2 P. acnes strains are more prevalent in “Decrease” cohort while the K1 strain is more prevalent in “Increase” cohort. FIG. 39 shows a change in P. acnes strains after treatment. The C2 P. acnes strain becomes more prevalent after benzoyl peroxide treatment. FIG. 40 shows a comparative functional analysis abased on response. A comparative functional analysis was done between those who responded to benzoyl peroxide and those who did not. FIG. 41 shows a change in P. acnes strains after benzoyl peroxide treatment. Functional mapping of P. acnes strains shows significant differences in lipase activity, antibiotic resistance, siderophores, and peptides/nickel transport system. FIG. 42 shows functional results of Manganese oxidation. Manganese oxidation is over-represented in the cohort that did not respond to benzoyl peroxide.

### Example 2—Product Recommendation

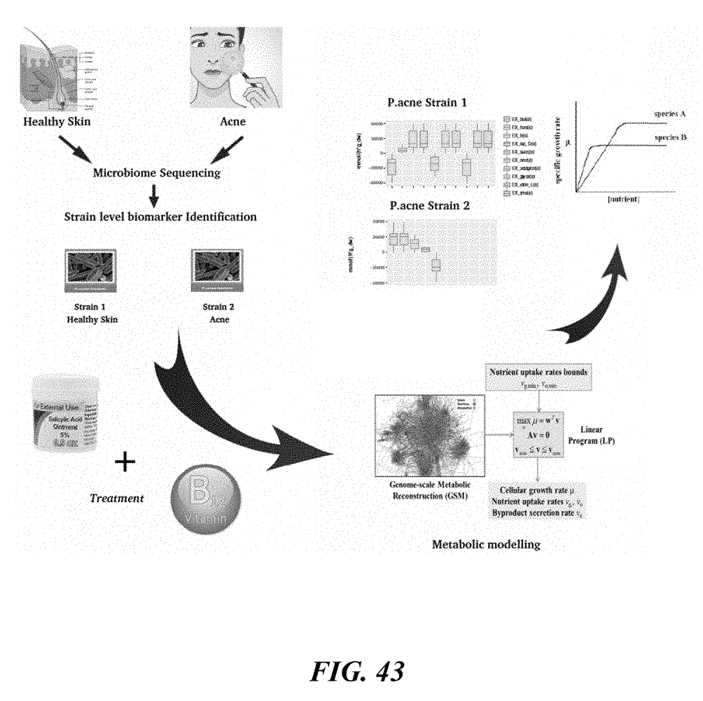

FIG. 43 shows an exemplary method for acne product recommendation. Strain level differences mapped between healthy and acne patients will be used to model the impact of existing or novel acne treatment options on manipulating undesired or desired bacterial strains.

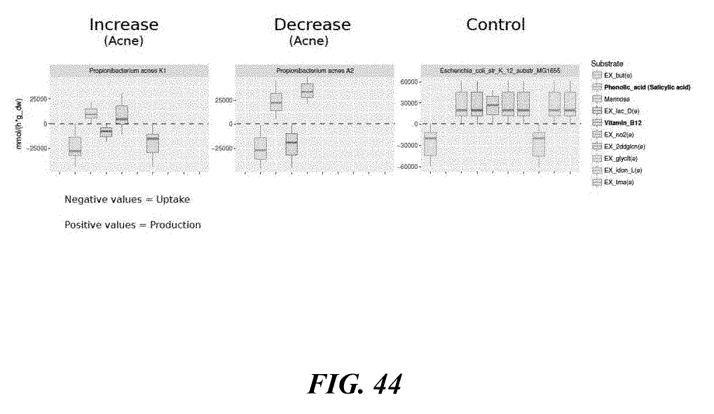

FIG. 44 shows result of metabolic modeling of good and bad strains based on different metabolites. Both K1 and A2 P. acne strains are capable of utilizing salicylic acid and also the production of vitamin B12 but the production of vitamin B12 is higher in Decrease group.

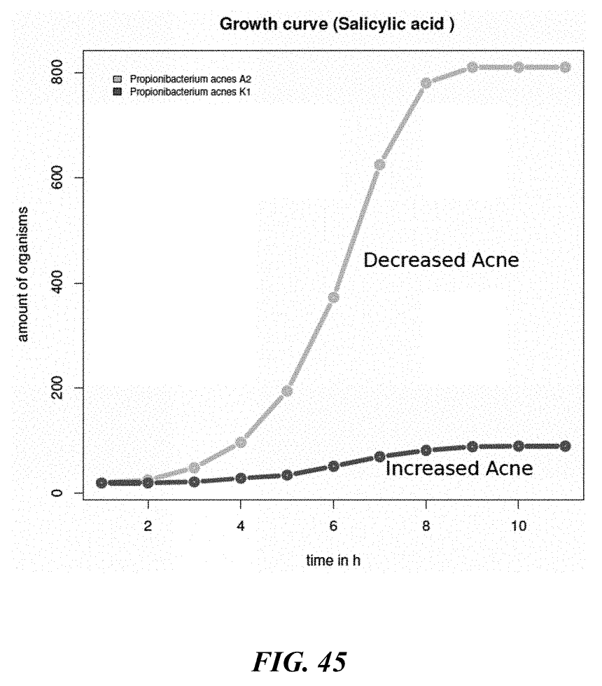

FIG. 45 shows the results of acne product recommendation based on metabolic modeling. K1 strain was more prominent in individuals which adversely responded to benzoyl peroxide treatment. The simulated growth curves show that salicylic acid could be a better treatment options for those people as it helps beneficial strains to take over.

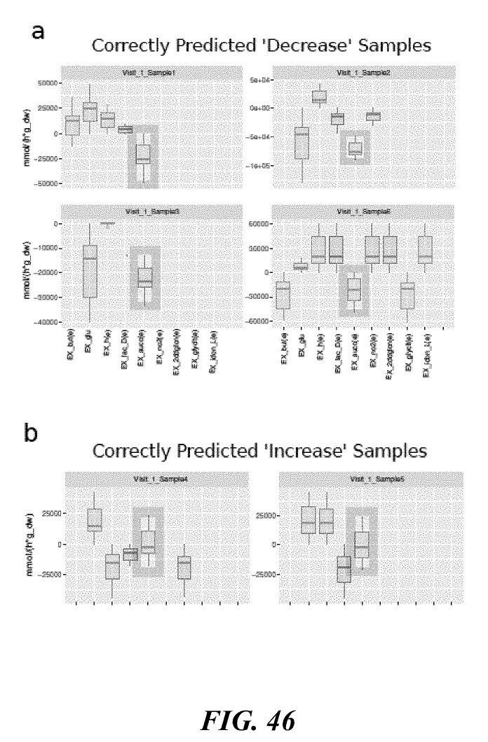

FIG. 46 shows a benzoyl peroxide uptake model based on microbiome profiles. A machine learning model was trained on all the subjects who were treated with benzoyl peroxide to identify the biomarkers that are responsible for poor or good response to benzoyl peroxide. The model was evaluated on subjects who were not exposed to benzoyl peroxide. As shown here in 6 examples, the model accurately predicted whether benzoyl peroxide can be consumed (negative value) or not by ‘Decrease’ or ‘Increase’ group respectively.

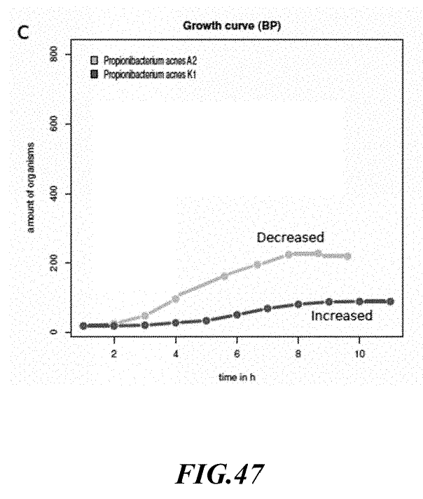

FIG. 47 shows the results of mapping metabolic models to strains differentially represented in benzoyl peroxide response groups. In correctly predicted Decrease sample from visit 1, the microbiome is capable of consuming benzoyl peroxide but when you see the growth Curve, the growth rate of good or healthy strain is higher in Decrease group. In correctly predicted Increase samples from visit 1, the microbiome is not capable of consuming benzoyl peroxide (value in Boxplot is near 0) and in growth curve plot you can see that mean growth of strain specific to increase group is very low on benzoyl peroxide.

### Example 3

A summary of results described herein include:
Number of non-control samples: 735
Number of non-control samples with a profile: 647
Samples worked as expected: 88%
Number of control samples: 100
Number of control samples with a profile: 6
Controls worked as expected: 94%

Among all the sites tested, cheek impacted the most upon benzoyl peroxide treatment. Benzoyl peroxide treatment does not impact the overall microbiome diversity. Patients that responded negatively to benzoyl peroxide had higher ratios of P. acnes in their profile. Certain P. acnes strains are associated with poor or good response to benzoyl peroxide.

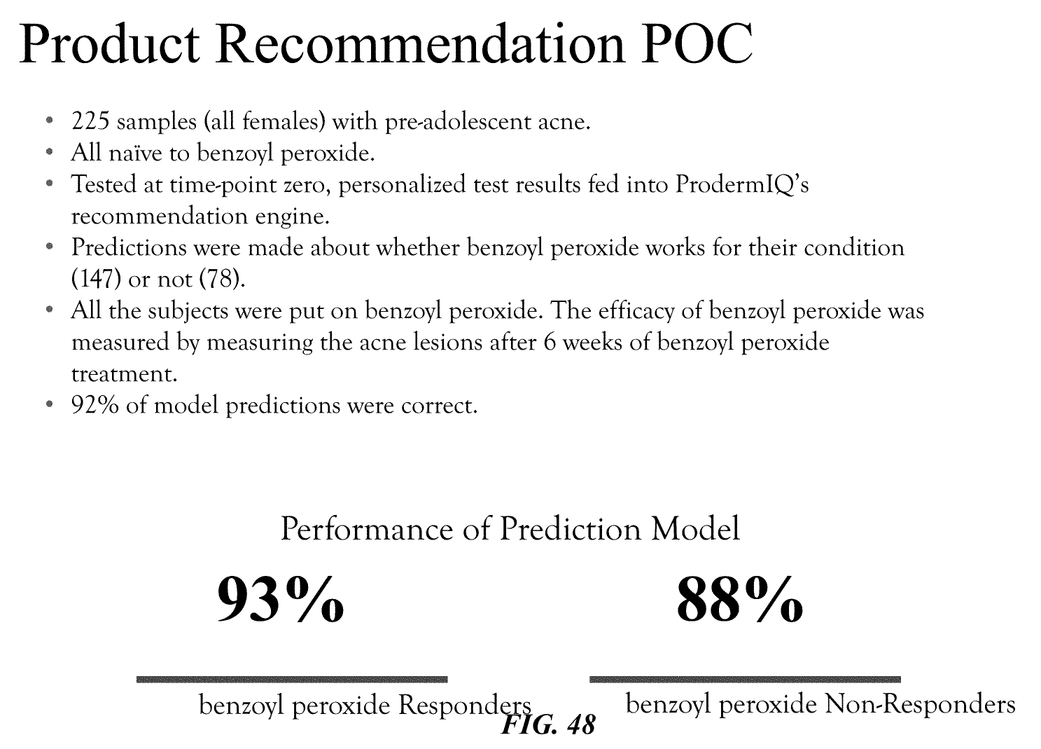

FIG. 48 shows a summary of the results of product recommendation proof of concept performed. 225 samples (all females) with pre-adolescent acne all were naïve to benzoyl peroxide. Tested at time-point zero, personalized test results fed into a recommendation engine. Predictions were made about whether benzoyl peroxide works for their condition (147) or not (78). All the subjects were put on benzoyl peroxide. The efficacy of benzoyl peroxide was measured by measuring the acne lesions after 6 weeks of benzoyl peroxide treatment. 92% of model predictions were correct.

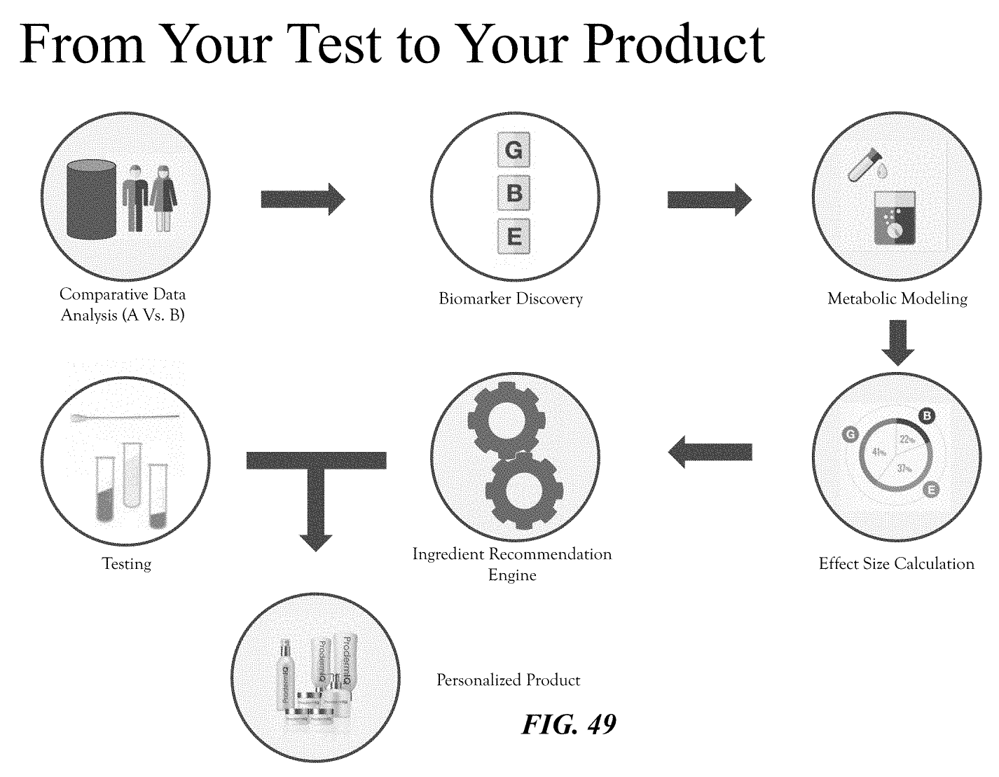

FIG. 49 shows the steps of an exemplary method from testing of an individual to the development of a custom treatment. In a first step, a Comparative Data Analysis (A Vs. B) occurs. In a second step, Biomarker Discover occurs. In a third step, Metabolic Modeling occurs. In a fourth step, an effect size calculation occurs. In a fifth step, results are run through an Ingredient Recommendation Engine. In an optional sixth step, testing occurs. In a seventh step, a personalized product is provided. It should be understood that in the above exemplary method, the steps may occur in the sequence described or, in some embodiments, one or more steps are carried out in a different order.

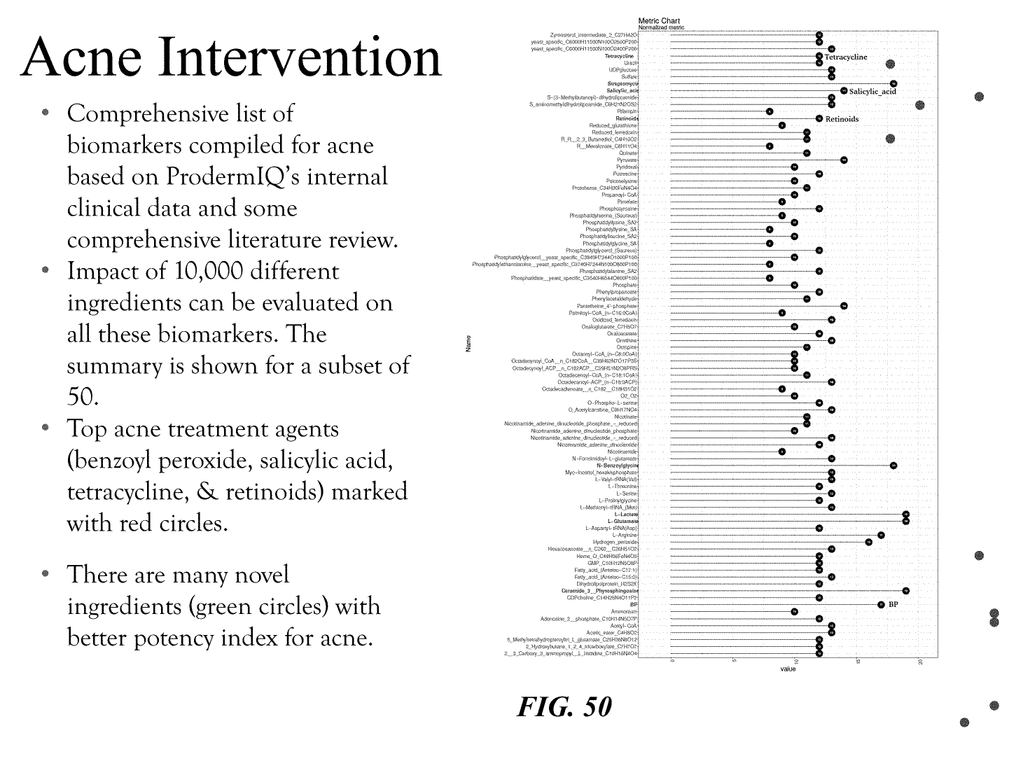

FIG. 50 shows the results of comprehensive biomarkers compiled for acne based on clinical data. Impact of 10,000 different ingredients can be evaluated on all these biomarkers. The summary is shown for a subset of 50. Top acne treatment agents (benzoyl peroxide, salicylic acid, tetracycline, & retinoids) marked with red circles. There are many novel ingredients (green circles) with better potency index for acne.

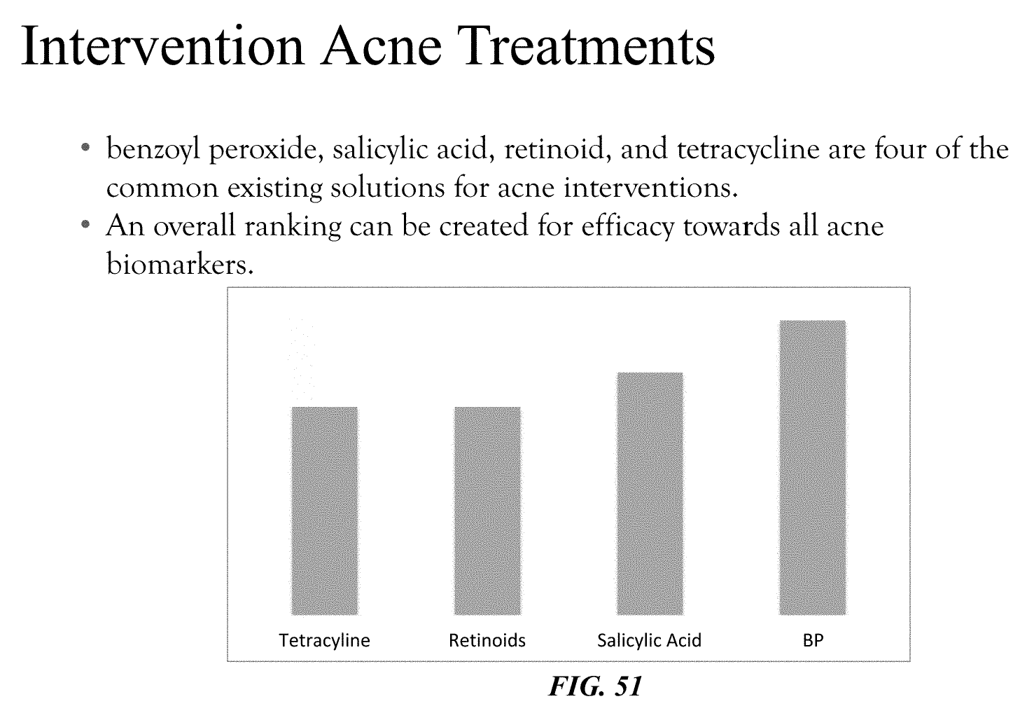

FIG. 51 shows a ranking of acne treatments. Benzoyl peroxide, salicylic acid, retinoid, and tetracycline are four of the common existing solutions for acne interventions. An overall ranking can be created for efficacy towards all acne biomarkers.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A computer based method for treating acne vulgaris in an individual, said method comprising:
obtaining a sample from said individual;
identifying a strain of P. acnes within said sample;
determining using a machine learning model that said strain of P. acnes is associated with a therapeutic response to benzoyl peroxide; and
treating said individual with said benzoyl peroxide when said strain of P. acnes is associated with said therapeutic response to said benzoyl peroxide.

2. The method of claim 1, wherein said machine learning model is generated by a convolutional neural network.

3. The method of claim 2, wherein said convolutional neural network is trained using supervised learning of labeled sample data taken from other individuals.

4. The method of claim 1, wherein said therapeutic response to benzoyl peroxide is determined using genomic sequencing of said strain of P. acnes.

5. The method of claim 1, wherein said strain of P. acnes associated with a therapeutic response to benzoyl peroxide is identified based on an analysis of a metabolism of said strain of P. acnes.

6. The method of claim 1, wherein said therapeutic response to benzoyl peroxide comprises a decrease of at least 10% in a population of said P. acnes within a treatment area.

7. The method of claim 1, wherein said strain of P. acnes is associated with a therapeutic response to benzoyl peroxide with an accuracy of at least 75%.

8. The method of claim 1, wherein said strain of P. acnes comprises a K4 strain.

9. The method of claim 1, wherein said strain said strain of P. acnes comprises an A2 strain.

10. A method of treating acne vulgaris in an individual, said method comprising:
providing benzoyl peroxide to said individual when a microbiome of a skin surface of said individual is found to comprise a strain of P. acnes selected from the group consisting of a K4 strain and an A2 strain.

11. The method of claim 10 comprising providing salicylic acid to said individual.

12. The method of claim 10, wherein said strain of P. acnes is associated with a therapeutic response to benzoyl peroxide.

13. The method of claim 12, comprising determining a metabolism of said strain of P. acnes and wherein said therapeutic response to benzoyl peroxide is based on said metabolism.

14. The method of claim 10, comprising instructing said individual to apply said benzoyl peroxide to an area of skin of said individual.

15. The method of claim 14, wherein a population of P. acnes is reduced by at least 50% on said area of said skin after said benzoyl peroxide is applied.

16. A method for analyzing a therapeutic efficacy of a treatment for acne vulgaris in an individual, said method comprising:
obtaining a sample from said individual;
determining a presence of P. acnes within said sample;
determining a sensitivity of said P. acnes to each one of a plurality of treatments;
ranking said each one of said plurality of treatments based on said sensitivity thereby identifying a most effective treatment and a second most effective treatment; and
generating a combination therapy that includes said most effective treatment and said second most effective treatment.

17. The method of claim 16, wherein said presence of said P. acnes is determined using genome sequencing on said sample.

18. The method of claim 16, wherein said sensitivity of said strain of said P. acnes is determined based on an analysis of a metabolism of said micro-organism.

19. The method of claim 16, wherein said sensitivity of said strain of said P. acnes is determined by a machine learning model that is generated by a convolutional neural network.

20. The method of claim 20, wherein said convolutional neural network is trained using supervised learning of labeled sample data taken from other individuals.
